SEQUENCE LISTING (25 sequences)

SEQ ID NO: 1 - protein, 42 aa, Homo sapiens [sequence omitted]

SEQ ID NO: 2 - DNA, 42 bp, Artificial Sequence
  Primer
cccgaagcttaccatggatgcagaattccgacatgactcagg

SEQ ID NO: 3 - DNA, 36 bp, Artificial Sequence
  Primer
gcgggatccaaaaatctggattgttgggttgataat

SEQ ID NO: 4 - DNA, 38 bp, Artificial Sequence
  Primer
cgactgagctcttaatcatttgtccatccttcatctgt

SEQ ID NO: 5 - DNA, 17 bp, Artificial Sequence
  Primer
actggcatggccttccg

SEQ ID NO: 6 - DNA, 18 bp, Artificial Sequence
  Primer
caggcggcacgtcagatc

SEQ ID NO: 7 - DNA, 25 bp, Artificial Sequence
  GAPDH probe sequence
ttcctacccccaatgtgtccgtcgt

SEQ ID NO: 8 - DNA, 20 bp, Artificial Sequence
  Primer
cctggagcagctgaatggaa

SEQ ID NO: 9 - DNA, 22 bp, Artificial Sequence
  Primer
ccgtcatctccatagggatctt

SEQ ID NO: 10 - DNA, 26 bp, Artificial Sequence
  IFN-b probe sequence
tcaacctcacctacagggcggacttc

SEQ ID NO: 11 - DNA, 23 bp, Artificial Sequence
  Primer
tgaacgctacacactgcatcttg

SEQ ID NO: 12 - DNA, 28 bp, Artificial Sequence
  Primer
gttattcagactttctaggctttcaatg

SEQ ID NO: 13 - DNA, 27 bp, Artificial Sequence
  IFN-g probe sequence
tttgcagctcttcctcatggctgtttc

SEQ ID NO: 14 - DNA, 23 bp, Artificial Sequence
  Primer
ctgcaagagacttccatccagtt

SEQ ID NO: 15 - DNA, 20 bp, Artificial Sequence
  Primer
aagtagggaaggccgtggtt

SEQ ID NO: 16 - DNA, 26 bp, Artificial Sequence
  IL-6 probe sequence
ccttcttgggactgatgctggtgaca

SEQ ID NO: 17 - DNA, 23 bp, Artificial Sequence
  Primer
caagaacatccaagcttgagtgt

SEQ ID NO: 18 - DNA, 20 bp, Artificial Sequence
  Primer
ttttgaccgcccttgagagt

SEQ ID NO: 19 - DNA, 25 bp, Artificial Sequence
  MIP2 probe sequence
cccactgcgcccagacagaagtcat

SEQ ID NO: 20 - DNA, 18 bp, Artificial Sequence
  Primer
tccaggcggtgcctatgt

SEQ ID NO: 21 - DNA, 21 bp, Artificial Sequence
  Primer
cgatcaccccgaagttcagta

SEQ ID NO: 22 - DNA, 27 bp, Artificial Sequence
  TNF-a probe sequence
cagcctcttctcattcctgcttgtggc

SEQ ID NO: 23 - DNA, 20 bp, Artificial Sequence
  Primer
ttcctcccaataccccttcc

SEQ ID NO: 24 - DNA, 23 bp, Artificial Sequence
  Primer
tgaagtcccggatacacagactt

SEQ ID NO: 25 - DNA, 23 bp, Artificial Sequence
  TNF-b probe
tgtgcctctcctcagtgcgcaga

(19) United States
(12) Patent Application Publication
(10) Pub. No.: US 2007/0264280 A1
(43) Pub. Date: Nov. 15, 2007

(54) Compositions and Methods for Treating Neurological Diseases

(76) Inventors: Howard J. Federoff, Rochester, NY (US); William J. Bowers, Webster, NY (US)

Correspondence Address: FISH & RICHARDSON PC, P.O. BOX 1022, MINNEAPOLIS, MN, 55440-1022, US

(21) Appl. No.: 10/578,561

(22) PCT Filed: Nov. 8, 2004
(86) PCT No.: PCT/US04/37511

§ 371 Date: Mar. 1, 2007

Related U.S. Application Data
(60) Provisional application No. 60/518,474, filed on Nov. 7, 2003.

(51) Int. Cl. A61K 38/17; A61K 39/00; A61P 25/00; A61P 25/28; C07H 21/04

(52) U.S. Cl. 424/193.1; 424/184.1; 514/12; 536/23.5

(57) ABSTRACT

The invention includes therapeutic compositions and methods useful in the treatment of neurodegenerative diseases, such as those characterized by accumulation of extracellular plaques. Such neurodegenerative diseases include Alzheimer's disease. The compositions of the invention include HSVAβ/TtxFC, which can be used to deliver effective therapeutic benefits to a patient without inducing inflammation.

This application claims the benefit of the filing date of U.S. Ser. No. 60/518,474, filed Nov. 7, 2003. For the purpose of any United States patent that may issue from the present application, the entire content of the prior provisional application is hereby incorporated by reference herein.

### STATEMENT AS TO FEDERALLY SPONSORED RESEARCH

Some of the work described here was funded by a grant from the National Institutes of Health (R01AG020204). The United States government may, therefore, have certain rights in the invention.

### TECHNICAL FIELD

The present invention relates to compositions and methods for treating patients who have been diagnosed as having a neurological disease. More particularly, the invention relates to compositions, including amplicon particles, that can be used to prevent Alzheimer's disease (AD) or to ameliorate or reverse the progression of AD and its attendant symptoms.

### BACKGROUND

Alzheimer's Disease is a neurodegenerative disorder associated with gradual functional decline, dementia and neuronal loss that is initiated in specific brain regions and advances in a disease-specific manner. Clinical hallmarks include progressive impairment in memory, judgment, decision-making, orientation to physical surroundings, and language, all of which vary considerably among afflicted individuals.

Although rare before the age of 50, AD affects nearly half of all people in the most rapidly growing portion of the U.S. population: those older than 85. As such, the current number of AD patients in the United States is expected to increase greatly in the coming years.

There is presently no known method of preventing AD. Current therapies are primarily supportive, such as those provided by a family member in attendance. Stimulated memory exercises on a regular basis have been shown to slow, but not stop, memory loss. A few drugs, such as tacrine (Cognex®), result in a modest temporary improvement of cognition but these drugs cannot stop the progressive dementia.

A hallmark of AD is the accumulation, in certain regions of the brain, of extracellular insoluble deposits called amyloid plaques, and abnormal lesions within neuronal cells called neurofibrillary tangles. When present, these plaques and tangles provide the only basis for a definitive diagnosis of AD.

The major components of amyloid plaques are the amyloid β-peptides, also called Aβ peptides, which consist of three proteins having 40, 42 or 43 amino acids, designated as the Aβ₁₋₄₀, Aβ₁₋₄₂, and Aβ₁₋₄₃ peptides, respectively. The amino acid sequences of the Aβ peptides are known; the sequence of Aβ₁₋₄₂ identical to that of Aβ₁₋₄₀, except that Aβ₁₋₄₂ contains two additional amino acid residues at its carboxyl terminus. Similarly, the amino acid sequence of Aβ₁₋₄₃ is identical to that of Aβ₁₋₄₂ except that Aβ₁₋₄₃ contains one additional amino acid at its carboxyl terminus. The Aβ peptides are thought to cause the nerve cell destruction in AD, in part, because they are toxic to neurons in vitro and in vivo.

The Aβ peptides are derived from larger amyloid precursor proteins (APP proteins), which consist of four proteins, designated as the APP₆₉₅, APP₇₁₄, APP₇₅₁, and APP771 proteins, which contain 695, 714, 751 or 771 amino acids, respectively. The different APP proteins result from alternative ribonucleic acid splicing of a single APP gene product. The amino acid sequences of the APP proteins are also known and each APP protein contains the amino acid sequences of the Aβ peptides.

Proteases, now referred to as secretases (e.g., BACE1) are believed to produce the Aβ peptides by recognizing and cleaving specific amino acid sequences within the APP proteins. Such sequence-specific proteases are thought to produce the peptides consistently found in plaques.

### SUMMARY

The present invention is based, in part, on our discovery that administration of a protein that naturally occurs within the plaques that form in AD brains can be used in conjunction with an adjuvant to improve the status of that disease in a well accepted animal model. A composition containing one or more naturally occurring Aβ proteins, or antigenic fragments or other biologically active variants thereof, can therefore be used to prevent, slow, or reverse the appearance of amyloid plaques and the onset or progression of AD or one or more of the signs and symptoms associated with neurological diseases such as AD. While the invention is not limited to proteins that work by any particular mechanism, we expect the compositions will induce or enhance an immune response in a patient to whom they are administered (e.g., a humoral immune response or an immune response that lacks a substantial cytotoxic T cell response (e.g., an immune response skewed toward a T helper cell response)). The invention features compositions that contain Aβ proteins (e.g., pharmaceutical compositions and kits), methods of making them, and methods of administering them to a patient (e.g., a human patient). Various embodiments are described further below.

The methods of the invention include methods of treating a patient who has been diagnosed as having a neurodegenerative disease characterized by extracellular plaques (e.g., amyloid plaques or plaques containing an Aβ protein) or the improper processing of APP. The methods can also be applied to a patient who is at risk of developing such a disease. Thus, the methods can be carried out on patients who are apparently healthy or who show no signs of AD as well as patients who have been diagnosed with AD. While all individuals are at some risk of developing Alzheimer's disease, some have a heightened risk due to, for example, advanced age or family history. Various mutations in the APP or Aβ proteins are known to be associated with a greater risk of AD (e.g., the Swedish mutation in the APP protein). The Dutch and Iowa mutations are associated with early onset AD and appear in both the APP protein and the Aβ proteins formed therefrom.

The precise way in which the treatment is carried out can vary so long as the patient receives a therapeutically effective amount of a composition that includes an Aβ protein (e.g., Aβ₁₋₄₀, Aβ₁₋₄₂, and/or Aβ₁₋₄₃ (e.g., a human Aβ protein)) or an antigenic fragment or other biologically active variant thereof (e.g., an Aβ protein that includes the Dutch or Iowa mutation). En one embodiment, the patient is treated by administering an Aβ protein or an antigenic fragment or other biologically active variant thereof (e.g., a substitution mutant). The protein can be presented as a linear epitope or engineered to offer a conformational epitope (e.g., a sequence that is conformationally constrained to better mimic the three-dimensional structure of the corresponding region on the antigen in vivo). For example, the Aβ proteins can be cyclized and may contain additional residues to join the C- and N-termini (e.g., a di- or tri-peptide linker). The immunogenicity of the engineered Aβ protein can be tested in numerous ways, including within an animal model of AD or in human volunteers. Alternatively, or in addition, the patient can be treated by administering a cell (e.g., an antigen presenting cell (APC) such as a dendritic cell) that expresses on its surface at least a portion of an Aβ protein or an antigenic fragment or other biologically active variant thereof. Alternatively, or in addition, the patient can be treated by administering a nucleic acid molecule that includes a sequence that encodes an Aβ protein or an antigenic fragment or other biologically active variant thereof For ease of reading, we do not continue to repeat the phrase “or an antigenic fragment or other biologically active variant thereof” at every opportunity. It is to be understood that where an Aβ protein can be used, one can also use an antigenic fragment or other biologically active variant thereof (i.e., a fragment, mutant, or other variant that confers a clinical benefit on a patient (e.g., a patient believed to have AD)). The term “Aβ protein” encompasses antigenic fragments and other biologically active variants thereof. These fragments and variants are described further below.

Regardless of the manner in which the Aβ protein is administered, it can be administered with an adjuvant. We may use the terms “adjuvant” or “molecular adjuvant” to refer to a substance (e.g., a protein or lipid) that amplifies a given response (e.g., an immune response or a clinical endpoint (e.g., an improvement in a cognitive function)) beyond the response that would typically occur in the absence of adjuvant. Amplification may be evident where, for example, on average, the immune response or the improvement in a patient's symptoms following the use of an adjuvant is as robust as that observed with a larger amount of antigen (i.e., Aβ protein) but no adjuvant. The adjuvant can be, for example, alum, tetanus toxoid (e.g., the C fragment of tetanus toxin (TtxFC)), keyhole limpet hemocyanin (KLH), aluminum hydroxide, aluminum phosphate, calcium phosphate, or an oil emulsion. Less traditional adjuvants include derivatives of muramyl dipeptide, monophosphoryl lipid A, liposomes, QS21, MF-59, and immunostimulating complexes (ISCOMS). The Aβ proteins of the invention (or cells expressing them or nucleic acids encoding them) can also be released in a controlled manner from biodegradable polymers (e.g., microspheres) and conjugated as protein-polysaccharide conjugates. See Gupta and Siber, Vaccine 13:1263-1276, 1995. Expressly excluded from the meaning of “adjuvant” are Aβ proteins and the immunomodulatory proteins (e.g., immunomodulatory cytokines) described below.

While we describe methods and routes of administration further below, we note here that the Aβ-containing compositions can be administered orally or parenterally (e.g., by an intravenous, subcutaneous, or intramuscular injection). For example, an Aβ-encoding nucleic acid can be placed within an expression vector such as a plasmid, virus, or amplicon particle (e.g., a herpes virus amplicon particle such as a herpes simplex virus (HSV) amplicon, which may be made in a helper-free system (e.g., an HSVhf amplicon as described in the U.S. patent application published under number 20030027322)). Amplicon particles are able to contain large amounts of nucleic acid. Accordingly, they can be used to co-express the Aβ protein, the adjuvant (where proteinaceous), and an immunomodulatory protein (described further below). Once made or provided, the Aβ-expressing vector can be injected into the patient. In the case of a herpes virus amplicon particle, components of the particle can be administered to the patient (as described further below). The amount delivered, whether delivered once or as a “prime” followed by one or more “boosters” (given for a limited time (e.g., once or twice) or over an extended period of time (e.g., about once every 2-6 months) will be sufficient to improve one or more symptoms of the neurodegenerative disease. For example, the composition will be of a type and amount sufficient to improve one or more of the following symptoms: impaired memory, impaired thinking (e.g., impaired abstract thinking or forgetfulness (manifested by, for example, misplacing objects)), disorientation, confusion, difficulty performing familiar tasks, changes in personality, changes in behavior, impaired judgment, impaired ability to follow directions, impaired communication skills (e.g., impaired language skills), impaired visual skills, impaired spatial skills, loss of motivation or initiative, change from normal sleep patterns, or any other relevant symptom of the neurological disease.

The Aβ protein and the molecular adjuvant can be admixed, chemically conjugated, or fused (e.g., into a recombinant fusion polypeptide). Alternatively, the Aβ protein and adjuvant can be maintained in separate containers and administered at the same time (or around the same time (e.g., sequentially)) by the same or different routes. Whether combined or provided separately, the compositions of the invention can be packaged with instructions (e.g., printed matter (e.g., written instructions or diagrams) and/or audio- and video instructions) as a kit. Optionally, the kit can provide paraphernalia for administering the composition(s) contained therein (e.g., syringes, needles, nebulizers, spray containers, alcohol swabs, and gauze or other dressing). For example, the invention features kits that include a vial containing one or more Aβ proteins and an adjuvant. The Aβ proteins and adjuvant may be concentrated or lyophilized and a diluent (e.g., a sterile, physiologically acceptable solution) may be provided in a separate vial. Alternatively, the Aβ proteins and adjuvant can be suspended and ready for use. Other components of the kits include immunomodulatory proteins, as described below.

Any of the methods described above can include administration of an immunomodulatory protein (i.e., a protein other than an Aβ protein or adjuvant). For example, in addition to administering an Aβ protein or an Aβ protein and an adjuvant, one can also administer an immunomodulatory cytokine that modulates the immune response to reduce the risk of inflammation (e.g., encephalitis). For example, one can also administer a chemokine such as RANTES; an interleukin such as interleukin-2 (IL-2), interleukin-4 (IL-4), interleukin-10 (IL-10), interleukin-12 (IL-12), interleukin-15 (IL-15) or interleukin-23 (IL-23); an interferon or growth factor (e.g., granulocyte macrophage colony stimulating factor (GM-CSF), tumor necrosis factor alpha (TNFα), or interferon-γ (IFNγ); an intracellular adhesion molecule (e.g., ICAM-1 (also known as CD54), ICAM-2, or ICAM-3); or a costimulatory factor that activates B or T cells (e.g., B7.1). The chemolcine can be one in the “C—C” family (e.g., MCP-1, MCP-2, MCP-3, DC-CK1, M-1α, MIP-3α, MIP-1β, MIP-3β); one in the “C—X—C” family (e.g., IL-8, SDF-1β, SDF-1α, GRO, PF-4 and MIP-2); one in the “C” family (e.g. lympotactin); or one in the “CX3C” family (e.g., fractalkine). As with the Aβ proteins and proteinaceious adjuvants, the immunomodulatory proteins can be administered as proteins per se (i.e., as pure or substantially pure proteins within the pharmaceutical composition), as proteins expressed on the surface of a cell, or as nucleic acids that are expressed in vivo as immunomodulatory proteins. Nucleic acid sequences encoding immunomodulatory proteins can be included in any of the expression vectors described herein and may be included in the same vector or type of vector as the sequence encoding the Aβ protein and/or the adjuvant. Similarly, while the immunomodulatory proteins can be full-length, naturally occurring proteins, they can also be biologically active variants thereof For example, one can administer a fragment or other mutant of an immunomodulatory protein (e.g., a substitution mutant) or a splice variant so long as the mutant or variant retains sufficient biological activity to confer a clinical benefit on the patient.

The invention also features compositions (e.g., pharmaceutically acceptable compositions) including any of those described above as suitable for use in the methods of treating a patient. For example, the compositions of the invention include Aβ proteins (e.g., Aβ₁₋₄₀, Aβ₁₋₄₂, and/or Aβ₁₋₄₃ and/or antigenic fragments or biologically active variants thereof) with, optionally, a molecular adjuvant (including any specifically described herein). Compositions that include an Aβ protein or an Aβ protein and an adjuvant can further include an immunomodulatory protein. Where the proteins are expressed from a delivery vehicle (e.g., a virus (e.g., a retrovirus or adenovirus), plasmid, or amplicon particle), that vehicle can constitute, or can constitute a part of, a composition of the invention (e.g., a pharmaceutical composition including a physiologically acceptable diluent (e.g., normal saline or phosphate-buffered saline (PBS))).

As noted above, the Aβ protein, a proteinaceious antigen, and/or an immunomodulatory protein may be expressed from the same delivery vehicle or same type of delivery vehicle as fused or unfused proteins. Accordingly, the invention encompasses delivery vehicles that include nucleic acid sequences encoding an Aβ protein and a sequence encoding a molecular adjuvant (e.g., TtxFC or KLH) and/or an immunomodulatory protein (e.g., IL-2, IL-12, or IL-23). The delivery vehicles may further include regulatory elements that facilitate the expression of the Aβ protein, a proteinacious adjuvant and/or an immunomodulatory protein.

In specific embodiments, the invention includes methods of treating a patient with a neurodegenerative disease associated with the presence of extracellular plaques (e.g., Alzheimer's disease) by administering to the patient (a) an amplicon plasmid or particle including an HSV origin of replication, an HSV cleavage/packaging signal, and a heterologous transgene expressible in a host cell, (b) one or more vectors that, individually or collectively, encode all essential HSV genes but exclude all cleavage/packaging signals, and (c) a vector encoding an accessory protein, in which the transgene encodes a therapeutic protein (e.g., a molecular adjuvant (e.g., TtcFC, KLH), an Aβ protein, or both), that improves one or more symptoms of the neurodegenerative disease.

In specific embodiments, the invention features compositions for use as medicaments in treating a patient with a neurodegenerative disease (e.g., Alzheimer's disease) characterized by extracellular plaques, in which the compositions include (a) an amplicon plasmid including an HSV origin of replication, an HSV cleavage/packaging signal, and a heterologous transgene expressible in a host cell, (b) one or more vectors that, individually or collectively, encode all essential HSV genes but exclude all cleavage/packaging signals, and (c) a vector encoding an accessory protein, in which the transgene encodes a therapeutic protein (e.g., a molecular adjuvant (e.g., TtxFC, KLH, Aβ, or both) that improves one or more symptoms of the neurodegenerative disease.

The invention additionally includes uses of compositions for the manufacture of a medicament for use in treating a patient with a neurodegenerative disease (e.g., Alzheimer's disease) characterized by extracellular plaques, in which the compositions includes (a) an amplicon plasmid including an HSV origin of replication, an HSV cleavage/packaging signal, and a heterologous transgene expressible in a host cell, (b) one or more vectors that, individually or collectively, encode all essential HSV genes but exclude all cleavage/packaging signals, and (c) a vector encoding an accessory protein, in which the transgene encodes a therapeutic protein (e.g., a molecular adjuvant (e.g. TtxFC or KLH), Aβ, or both) that improves one or more symptoms of the neurodegenerative disease.

Our studies indicate that the treatment methods described herein will benefit at least some patients in ways that are not readily achieved by present treatments or therapies. For example, the present treatment could slow or stop the accumulation of Aβ plaques and may even reverse their size or number, thus providing substantial and prolonged improvement of the symptoms of AD. Moreover, the present treatment is expected to accomplish this desirable effect in humans without causing substantial inflammation in the patient's brain. Thus, the present methods may be more effective and safer than current methods. Furthermore, by providing model organisms, this invention allows the further development of treatments for Alzheimer's disease.

Although methods and materials similar or equivalent to those described herein can be used in the practice or testing of the present invention, suitable methods and materials are described below. All publications, patent applications, patents, and other references mentioned herein are incorporated by reference in their entirety, including U.S. patent application Ser. Nos. 09/997,848 and 10/296,551, and U.S. Provisional Patent Application Ser. Nos. 60/250,079, 60/385,230, 60/442,030, and 60/480,112, especially as their disclosures relate to making and using HSV amplicons. The details of one or more embodiments of the invention are set forth in the accompanying drawings and is the description below. Other features, objects, and advantages of the invention will be apparent from the description and drawings, and from the claims.

### BRIEF DESCRIPTION OF THE DRAWINGS

FIGS. 1A and 1B are schematic representations of amplicon vectors and the study design. FIG. 1A depicts two novel HSV amplicons plasmids that were constructed: one that expresses the Aβ₁₋₄₂ peptide derived from APP (HSVAβ), and another that expresses Aβ₁₋₄₂ fused in-frame at the C-terminus with tetanus toxin Fragment C (HSVAβ/TtxFC). A previously described amplicon that expressed β-galactosidase (Geller and Breakefield, Science 241:1667-9, 1988; HSVlac) served as a control vector. All amplicons were packaged using a previously described helper virus-free method (Bowers et al., Gene Ther. 8, 2001). FIG. 1B depicts how each packaged vector (1×10⁵ transduction units) was delivered subcutaneously (s.c.) to APP_{Swe}-overexpressing transgenic mice (Hsiao et al., Science 274(5284):99-102, 1996; Tg2576) or non-transgenic littermates at 4-8 weeks of age. Amplicons were administered monthly to each animal three times, and humoral assessments were performed one week post-injection and subsequently at one-month intervals. Antibody isotype analysis was performed on sera obtained at the 4-month timepoint. Treated mice were sacrificed at 11 months of age, at which time end-point histological and stereological analyses were performed.

FIGS. 2A and 2B are a pair of graphs showing that HSV amplicon-delivered Aβ antigens elicit marked humoral responses. Helper virus-free HSV amplicons (1×10⁵ transduction units) were delivered subcutaneously to APP_{Swe}-overexpressing transgenic mice (Tg2576) at six weeks of age. Serum was obtained from each vaccinated mouse according to the schema illustrated in FIG. 1B, and 1:256 dilutions were analyzed by ELISA. Levels of antigen-specific antibodies arising from each vaccination were corrected using serum isolated from HSVlac control mice, and are expressed as “Corrected Absorbance @ 450 nm” for a subset of timepoints. FIG. 1A is a series of photomicrographs demonstrating that analysis of sera isolated from vaccinated mice by α-Aβ ELISA showed that both amplicon-expressed Aβ immunogens were capable of eliciting Aβ-specific humoral responses. Responses induced by the Aβ/TtxFC immunogen elevated at most assay time points and were more durable than those elicited by HSVAβ. FIG. 2B is a graph showing that analysis of α-TtxFC antibody titers by ELISA; TtxFC responses were specifically generated only in HSVAβ/TtxFC-vaccinated mice. Error bars represent standard deviation, while “*” indicates statistical significance (P<0.05) between HSVAβ/TtxFC and HSVβ values at same timepoint.

FIGS. 3A-3F are graphs showing that the antibodies elicited by HSV amplicon-delivered Aβ/TtxFC are more Th2-like and more mature than those elicited by HSVAβ. Isotypes of α-Aβ antibodies were determined by ELISA using sera obtained from vaccinated Tg2576 mice at the 4-month post-treatment timepoint. Levels of Aβ-specific antibody isotypes arising from each vaccination were corrected using serum isolated from HSVlac control mice, and are expressed as “Corrected Absorbance @ 450 nm”. Error bars represent standard deviation. Marked differences in isotypes were observed between animals receiving the two Aβ immunogen forms. HSVAβ-treated mice harbored exclusively α-Aβ antibodies of the IgM class while the HSVAβ/TtxFC-immunized Tg2576 mice produced antibodies primarily of the IgG1 isotype, with detectable levels of the IgA class. In addition, there existed κ light chain bias in the α-Aβ antibody pool obtained from HSVAβ/TtxFC-injected mice.

FIG. 4 is a graph showing that HSVAβ-vaccinated mice exhibit enhanced pro-inflammatory molecule expression profiles in the hippocampus, as assessed by quantitative real-time RT-PCR. Tg2576 and non-transgenic littermates received equal numbers of virions (1×10⁵ transducing units) subcutaneously at 8 and 12 weeks of age and animals were sacrificed one week after the final injection. Total RNA was isolated from microdissected hippocampus from one hemisphere of each mouse (n=74 per group). Levels of various pro-inflammatory molecule transcripts were determined using quantitative “real-time” RT-PCR, and values expressed as relative transcript level (mean±standard deviation) normalized to levels of a GAPDH internal control target. Injection of Tg2576 mice with HSVAβ led to a specific up-regulation of IFN-β (A), IFN-γ (B), IL-6 (C), MIP-2 (D), and TNF-α (E) transcripts as compared to HSVlac-vaccinated Tg2576 mice. HSVAβ-treated non-transgenic mice did not exhibit these enhanced pro-inflammatory transcript profiles. Assessment of TNF-β (F) expression determined a positive trend in HSVAβ-vaccinated Tg2576 mice but the difference as compared to the HSVlac-treated cohort did not reach significance. Similar analyses of HSVAβ/TtxFC-treated mice of either genotype showed only a statistically significant up-regulation of the chemoline MIP-2, while all other markers in the hippocampus of these animals remained similar to HSVlac controls. Error bars represent standard deviation, while “*” indicates statistical significance (P<0.05) between HSVAβ or HSVAβ/TtxFC and HSVlac control values.

FIGS. 5A and 5B are a pair of graphs showing that HSVAβ/TtxFC-treated Tg2576 mice exhibit altered plaque morphology and reduced numbers of small Aβ-immunopositive deposits. To qualitatively and quantitatively assess brain-harbored Aβ burden, Tg2576 mice and non-transgenic littermate controls (Non-Tg) receiving HSVlac (n=3) or HSVAβ/TtxFC (n=4) were sacrificed at 11 months of age, perfused, and brains processed for immunocytochemical analysis. FIG. 5A is a graph of representative immunocytochemical staining with the α-Aβ antibody 6E10, of brain sections highlighted marked differences in the appearance of Aβ deposits between HSVlac- and HSVAβ/TtxFC-vaccinated Tg2576 mice. Background staining in Non-Tg mice is also shown for comparison purposes. Brain-harbored Aβ deposits appeared qualitatively different in HSVlac-treated Tg2576 mice than in HSVAβ/TtxFC-immunized counterparts. FIG. 5B is a graph showing quantitative morphometric analyses performed to enumerate differences in brain Aβ plaque burden in 11 month-old Tg2576 mice. The numbers of 6E10-immunopositive deposits were determined for each of three deposit area ranges (50 μm² to 200 μm², 200 μm² to 500 m², and deposit areas >500 μm²). HSVAβ/TtxFC vaccination resulted in a decrement in numbers of deposits occupying the smallest area. Error bars represent standard deviation, while “*” indicates statistical significance (P<0.05) between HSVAβ/TtxFC and HSVlac values in same range of deposit size.

FIG. 6 is a table that summarizes mouse survival data.

### DETAILED DESCRIPTION

While the etiology of AD is presently unknown, substantial experimental and pathological data indicate that proteins cleaved from the amyloid precursor protein (APP) are key participants in pathogenesis. These cleavage products, the Aβ peptides, undergo a process termed fibrillogenesis, which leads to the formation of a series of structural intermediates that exhibit differential neurotoxicities. Accumulation of these pathogenic Aβ peptides via enhanced production and/or formation of proto-fibrillar intermediates leads to synaptic dysfunction and, eventually, to neuronal cell death (Hardy et al., Nat. Neurosci. 1(5):355-358, 1998; Lambert et al., Proc. Natl. Acad. Sci. USA 95(11):6448-6453, 1998; Miravalle et al., J. Biol. Chem. 275(35):27110-27116, 2000). Data generated in animal models suggests that attempting to raise an immunological response against the Aβ protein leads to inflammation in the brain that can cause severe damage, and even death, in the subject. The current invention is based on an approach that delivers therapeutic benefits without such adverse side-effects (or with a tolerable level of adverse side-effects). One approach employs an Aβ-based composition (e.g., a herpes virus amplicon particle or other delivery vehicle(s) that express an Aβ protein and, optionally, an adjuvant and immunomodulatory protein to skew the immune response away from a cytotoxic inflammatory T cell response). Such compositions, upon administration to a patient, can elicit an immune response against pathogenic forms of the Aβ peptide, thereby inhibiting Aβ accumulation and/or leading to the dissolution of Aβ-containing aggregates. As noted above, this response can occur without potentiating brain inflammation. Utilizing virus vector-based vaccination provides one means to elaborate Aβ-specific immune responses that can be optimally tailored to Alzheimer's disease. While the invention is not limited to particular delivery vectors, we expect our vectors, including the herpes virus amplicon particles, will be more predictable and efficacious than conventional peptide/adjuvant paradigms. Helper virus-free herpes simplex virus (HSV) amplicon vectors elicit vigorous transgene product-specific immune responses in vivo (Hocknell et al., J. Virol. 76(11):5565-5580, 2002; Wang et al., Vaccine 21(19-20):2288-22897, 2003; Willis et al., Hum. Gene Ther. 12(15):1867-1879, 2001). Given its ease of manipulation, absence of immunosuppressive viral genes, ability to efficiently transduce antigen presenting cells, and large transgene capacity, the amplicon is a well-positioned platform on which to build an Aβ-directed AD therapeutic. Any of the compositions of the invention, including those containing amplicon particles, can be used to test Aβ antigens with differential immune activities in an animal model (e.g., a mouse model) of AD. Such models are useful for determining the mechanisms underlying vaccine-induced brain inflammation, and for analyzing various combinations of Aβ proteins, adjuvants, and immunomodulatory proteins.

The invention is not limited to compositions that treat or prevent neurological disease (e.g., AD) by any particular mechanism, and a variety of mechanisms may underlie the ability of active Aβ-directed immunization to reduce amyloid burden. For example, anti-Aβ antibodies may directly inhibit and potentially reverse Aβ fibrillogenesis by assisting in plaque solubilization (Bacskai et al., J. Neurosci. 22(18):7873-7878, 2002). If correct, then an ideal treatment should induce a strong antibody response, mainly of isotypes that can traverse the blood-brain barrier. Helper T cell function, normally required for an effective antibody response, should in that case be as limited as possible to Th2-biased responses, as a strong Th1 response carries the risk of inducing a local inflammatory response to the Aβ antigen if T cells penetrate the blood/brain barrier for any reason (Becher et al., Glia 2(4):293-304, 2000), and compositions of the invention can elicit such a response. In addition, anti-Aβ antibodies may act to capture soluble Aβ, thereby preventing its participation in seeding of extracellular plaques (DeMattos et al., Proc. Natl. Acad. Sci. USA 98(15):8850-8855, 2001).

Complement activation by antibody/Aβ antigen complexes may have either useful or deleterious effects in the context of AD immunotherapy—complement deposition may assist in the dissolution of antigen/antibody complexes that develop in the plaques as a result of antibody binding (Miller and Nussenzweig, Proc. Natl. Acad. Sci. USA 72(2):418-422, 1975). In fact, amyloidogenic mice devoid of the complement component C3 exhibit markedly enhanced neurodegeneration and amyloid deposition, supporting an important role of complement activation and innate immune responses in protection from Aβ-mediated neurotoxicity (Wyss-Coray et al., Proc. Natl. Acad. Sci. USA 99(16):10837-10842, 2002). Bard et al. have demonstrated that antibody isotypes proficient in activating phagocytic cells through Fc receptors were very effective in dissolving amyloid deposits in a mouse model of AD (Bard et al., Proc. Natl. Acad. Sci. USA 100(4):2023-2028, 2003).

It is important to evaluate the antibody isotypes that are induced by candidate compositions, as this information may provide insight into the contribution of Th1 and Th2 T cells to the anti-Aβ immune response. Isotype analysis of anti-Aβ specific antibodies generated in Tg576 mice receiving HSVAβ or HSVAβ/TtXFC demonstrated the fundamental roles that antigen context and molecular adjuvants play in the generation of antibody isotypes. Molecular adjuvants, like TtxFC, appear to assist in expansion and maturation of humoral immune responses (Lu et al., Infect. Immun. 62(7):2754-60, 1994). In studies related to the current invention, HSVAβ-vaccinated mice failed to switch from an immature Ig isotype to one considered more mature, while HSVAβ/TtxFC-treated mice effectively generated anti-Aβ antibodies of the IgG1 class. Because IgG1 antibodies arise as a result of Th2 T cell participation, HSVAβ/TtxFC vaccination appears to have biased the anti-Aβ humoral response by activating the Th2 arm.

Vaccine antigen-mediated stimulation and T cell-driven proliferation and differentiation of naive B cells results in the generation of antigen-specific memory B cells and plasma cells carrying somatically mutated immunoglobulin loci (Banchereau et al., Annu. Rev. Immunol. 12:881-922, 1994; Manz et al., Nature 3 88(6638):133-134, 1997; Slifka et al., Immunity 8(3):363-372, 1998), and generation of optimal B cell memory is a vital consideration when designing an Aβ-based therapeutic treatment for humans. Bemasconi et al. have demonstrated that activation of the memory B cell component is required for long-lasting therapeutic action (Bernasconi et al., Science 298(5601):2199-2202, 2002). Antigen-dependent “short-term serological memory” mediated by plasma cells lasts only a few months, while “long-term serological memory” requires antigen-independent polyclonal activation and differentiation of memory B cells. In those experiments, co-delivery of adjuvant-like proteins during or after antigen-specific vaccination led to a population-wide activation and differentiation of memory B cells with polyclonal specificities. Mouse models of AD demonstrate there is a memory response participating in the protective action and durability of the HSVAβ/TtxFC therapeutic treatment.

Immunization of Tg2576 mice with HSVAβ led to a high rate of mortality, death that occurred approximately 1-2 weeks following the second vector inoculation. Subcutaneous injection of HSVAβ induced an adverse reaction, probably encephalitis, specifically within the brains of these mice. Quantitative real-time RT-PCR analysis of RNA isolated from the hippocampus was employed as a correlate of a hyperinflammatory CNS state. These experiments revealed a statistically significant enhancement of pro-inflammatory molecule expression (IFN-β, IFN-γ, IL-6, MIP-2, and TNF-α) in HSVAβ-vaccinated Tg2576 mice.

TNF-α is a potent cytokine produced by astrocytes, microglia, and neurons following pathological stress (Perry et al., Curr. Opin. Neurobiol. 5(5):636-641, 1995). TNF-α promotes infiltration of inflammatory cells, modulates MHC class I expression (Lavi et al., J. Neuroimmunol. 18(3):245-253, 1988), and induces the production of other cytokines in the brain (Das and Potter, Neuron 14(2):447-56, 1995; Nilsson et al., Neurochem. Int. 39(5-6):361-370, 2001). IFN-γ is expressed by activated Th1 T lymphocytes and NK cells (Boehm et al., Annu. Rev. Immunol. 15:749-795, 1997; Farrar and Schreiber, Annu. Rev. Immunol. 11:571-611, 1993), and has been shown to activate microglial cells, up-regulate MHC class II antigens, promote leukocyte adhesion, and increase nitric oxide production by promoting the transcription of iNOS in the brain (Colton et al., J. Neuroimmuzol. 40(1):89-98, 1992; Frei et al., Eur. J. Immunol. 17(9):1271-1278, 1987; Hewett et al., Neurosci. Lett. 164(1-2):229-232, 1993; Hickey and Kimura, Science 239(4837):290-292, 1988). IFN-β stimulates macrophages and NK cells, possesses antiviral activity, and modulates MHC class I expression. The pro-inflammatory cytokine, IL-6, is secreted by stimulated monocytes and macrophages as well as by astrocytes, microglia, and Th2 T cells (Akira et al., Adv. Immunol. 54:1-78, 1993; Gadient and Otten, Prog. Neurobiol. 52(5):379-390, 1 997). In the CNS, IL-6 triggers a cytoline cascade (Di Santo et al., Brain Res. 740(1-2):239-244, 1996) and modulates the activation of infiltrating T cells (Taga and Kishinaoto, Annu. Rev. Immunol. 15:797-819, 1997). MIP-2 is a chemokine that induces the migration and margination of neutrophils and is typically produced by macrophages. Elaboration of all these major pro-inflammatory cytokines within the CNS of Tg2576 mice was observed as a result of HSVAβ vaccination. Given the potency of these molecules, up-regulation of all or even a subset of them would be expected to impart profound effects on immune cell activation, neuronal and glial function, and cellular viability in Tg2576 mice (Giovannini et al., Neurobiol. Dis. 11 (2):257-274, 2002; Hauss-Wegrzyniak et al., Exp. Neurol. 176(2):336-341, 2002). Understanding the mechanism whereby the HSVAβ vaccination paradigm specifically induces such a marked pro-inflammatory response in the brain could provide valuable insight into the severe inflammatory events observed in patients receiving the experimental AN-1792 vaccine (Orgogozo et al., Neurology 61(1):46-54, 2003).

The HSV-1 amplicons employed in the studies below are encompassed by the invention and can be used to express any combination of the Aβ proteins, adjuvants, and/or immunomodulatory proteins described herein. These delivery vehicles possess a number of advantages over other gene delivery platforms. First, the amplicon is not a live virus (as are vaccinia, canarypox, etc.) and therefore, has an inherently safer in vivo profile. Second, compared to DNA delivery systems or most virus-based vectors, expression is directed from multiple episomal copies within each transduced cell, and the genome is maintained for a prolonged period in non-dividing cells such as antigen presenting cells (APCs). Third, the transgene size limit is larger (<130 kb; (Wade-Martins et al., Nucleic Acids Res 27(7):1674-82, 1999; Wade-Martins et al., Mol. Ther. 7(5):604-612, 2003; Wade-Martins et al., Nature Biotechnol. 19(11):1067-1070, 2001) than many other viral vectors providing an opportunity to co-express factors with known immunomodulating activity. And, the lack of encoded viral genes avoids the effects that wild-type herpes viruses typically use to evade the immune system, such as downregulation of MHC expression and antigen processing, and inhibition of dendritic cell maturation (Salio et al., Eur J. Immunol. 29(10):3245-53, 1999; Thomas and Rouse, Immunol Res 16(4):375-86, 1997).

Compositions: The invention includes compositions that can be used to treat Alzheimer's disease and other disorders associated with unwanted production of Aβ proteins. These compositions can include any of the Aβ proteins described herein (e.g., a human Aβ protein) and an adjuvant and/or immunomodulatory protein in a lyophilized form or suspended in a diluent suitable for administration to a patient (e.g., a buffered solution (e.g., PBS)). Also included are nucleic acid molecules that encode the Aβ proteins described herein (e.g., nucleic acid molecules that are isolated from the nucleic acids they are flanked by in a natural setting), vectors containing those nucleic acids (e.g., the amplicon particles), and cells (e.g., cells isolated from an intact animal) that express the Aβ proteins (e.g., antigen presenting cells such as dendritic cells).

More specifically, the compositions can include Aβ₁₋₄₀, Aβ₁₋₄₂, Aβ₁₋₄₃, HSVAβ, and HSVAβ/TtxFC. The Aβ proteins can have a sequence found in nature, including wild-type, Dutch, and Iowa mutations. For example, the Aβ₁₋₄₂ protein can have the sequence (from the N- to the C-terminus): Asp-Ala-Glu-Phe-Arg-His-Asp-Ser-Gly-Tyr-Glu-Val-His-His-Gin-Lys-Leu-Val-Phe-Phe-Ala-Glu-Asp-Val-Gly-Ser-Asn-Lys-Gly-Ala-Ie-Ile- Gly-Leu-Met-Val-Gly-Gly-Val-Val-Tle-Ala (SEQ ID NO:26). The sequences of Aβ proteins are known in the art (as are the sequences of the proteinaceous adjuvants and immunomodulatory proteins described herein). The nucleic acid molecules encoding the proteins described herein (i.e., the Aβ proteins, proteinaceous adjuvants, and immunomodulatory proteins) can be naturally occurring or may be degenerate variants.

Alternatively, or in addition (as the compositions can contain more than one type of Aβ protein), the proteins can be antigenic variants of an Aβ protein. For example, the compositions can include one or more fragments of an Aβ protein having, for example, about 10-20 (e.g., 12, 15, or 18), 10-25 (e.g., 17, 19, 21, or 23), 10-30 (e.g., 11, 13, 20, 25, 26, 27, 28, 29, or 30), or 30-40 (e.g., 32, 33, 34, 35, 36, 37, 38, or 39) residues. The sequence of the Aβ protein, regardless of length, can also vary from that found in nature. For example, the sequence may contain one or more substituted residues (e.g. conservative amino acid substitutions) so long as the protein remains capable of eliciting a desirable immune response against an amyloid plaque. The sequence may in fact be quite different from that of a naturally occurring Aβ protein.

The compositions of the invention can include, as noted above, molecular adjuvants capable of assisting in the expansion and maturation of humoral immune responses; see Lu et al., Infect. Immun. 62(7):2754-2760, 1994), and biologically active fragments thereof, as well as any of the various vehicles (e.g., an amplicon particle (e.g., an HSV-1 amplicon), viral vector (e.g., retroviral or adenoviral vector), plasmid, YAC, or BAC) that can be employed to deliver the former compositions to targeted tissues (e.g., brain tissue) and cells.

Aβ proteins, adjuvants, and immunomodulatory proteins may be about 60%, 75%, 80%, or even 90% or more (e.g., 95, 96, 97, 98, or 99%) identical to their naturally occurring counterparts and retain one or more of the biological activities of the full-length polypeptides of the invention. Such comparisons are generally based on an assay of biological activity in which equal concentrations of the polypeptides are used and compared. The comparison can also be based on the amount of the polypeptide required to reach 50% of the maximal stimulation obtainable.

As noted, functionally equivalent or biologically active variants (polypeptides or nucleic acids) can be those, for example, that contain additional or substituted components (amino acid residues or nucleotides, respectively). Substitutions may be made on the basis of similarity in polarity, charge, solubility, hydrophobicity, hydrophilicity, and/or the amphipathic nature of the residues involved. For example, a functionally equivalent polypeptide is one in which 10% or fewer of the amino acids in a full-length, naturally occurring polypeptide are replaced by conservative amino acid substitutions, and the functionally equivalent polypeptide maintains at least 50% of the biological activity of the full-length polypeptide. Conservative amino acid substitution refers to the substitution of one amino acid for another amino acid of the same class (e.g., valine for glycine, arginine for lysine, etc.).

Polypeptides that are functionally equivalent to polypeptides of the invention can be made using random mutagenesis on the encoding nucleic acids by techniques well known to those skilled in the art. It is more likely, however, that such polypeptides will be generated by site-directed mutagenesis (again using techniques well known to those skilled in the art). These polypeptides may have increased functionality or decreased functionality, but can be used to practice the methods of the invention as long as they remain capable of eliciting a desired immune response and/or inhibiting the onset or progression of a sign or symptom of neurological disease (e.g., AD).

Mutations within the coding sequence of nucleic acid molecules of the invention can be made to generate variant genes that are better suited for expression in a selected host cell. For example, N-linked glycosylation sites can be altered or eliminated to achieve, for example, expression of a homogeneous product that is more easily recovered and purified from yeast hosts that are known to hyperglycosylate N-linked sites. To this end, a variety of amino acid substitutions at one or both of the first or third amino acid positions of any one or more of the glycosylation recognition sequences which occur, and/or an amino acid deletion at the second position of any one or more of such recognition sequences, will prevent glycosylation at the modified tripeptide sequence (see, for example, Miyajima et al., EMBO J., 5:1193, 1986).

The polypeptides of the invention can be expressed from the same delivery vehicle (particularly where that vehicle has the capacity of an amplicon particle) and may be fused to one another or to another polypeptide (e.g., a marker, a polypeptide that facilitates purification, or a polypeptide that increases the circulating half-life of a protein to which it is attached). For example, the polypeptide can be fused to a hexa-histidine tag to facilitate purification of bacterially expressed protein or a hemagglutinin tag to facilitate purification of protein expressed in eukaryotic cells. The Aβ protein can be fused to all or part of an albumin polypeptide or an immunoglobulin (e.g., the Fc region of an IgG) in order to increase it's circulating half life.

A fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA, 88: 8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

If desired, the polypeptides of the invention can be chemically synthesized (for example, see Creighton, “Proteins: Structures and Molecular Principles,” W. H. Freeman & Co., NY, 1983), or, perhaps more advantageously, produced by recombinant DNA technology as described herein. For additional guidance, skilled artisans may consult Ausubel et al., “Current Protocols in Molecular Biology, Vol. I,” Green Publishing Associates, Inc., and John Wiley & Sons, Inc., NY, 1989, Sambrook et al. (“Molecular Cloning, A Laboratory Manual,” Cold Spring Harbor Press, Cold Spring Harbor, NY, 1989), and, particularly for examples of chemical synthesis Gait, M. J. Ed. (“Oligonucleotide Synthesis,” IRL Press, Oxford, 1984).

The delivery vehicles can include any of those used routinely in the art (e.g., plasmids with regulatory elements and the viral vectors described herein). The herpes virus amplicons (described further below) can be constructed using published U.S. patent applications as a guide (see, e.g., 20030027322, 20040105844, and 2004157299).

Methods of Administration: There are a variety of methods for successfully administering the compositions of the invention to a patient. They can be delivered to a patient orally or parenterally (e.g., by injection (e.g., intramuscular, intravenous, or subcutaneous injection)). The compositions can also be delivered to cells (e.g., cells within a patient or in tissue culture) using any of the gene delivery methods known in the art. These methods include direct injection, high-speed bombardment (e.g., by gene gun), and lipofection.

Amplicons: Helper virus-free systems for packaging herpesvirus particles, including those described herein, include at least one vector (herein, “the packaging vector”) that, upon delivery to a cell that supports herpesvirus replication, will form a DNA segment (or segments) capable of expressing sufficient structural herpesvirus proteins that a herpesvirus particle will assemble within the cell. When the particle assembles, amplicon plasmids that may also be present, can be packaged within the particle as well. In packaging systems that employ helper viruses, amplicon plasmids rely on the helper virus function to provide the replication machinery and structural proteins necessary for packaging amplicon plasmid DNA into viral particles. Helper packaging function is usually provided by a replication-defective virus that lacks an essential viral regulatory gene. The final product of helper virus-based packaging contains a mixture of varying proportions of helper and amplicon virions. Recently, helper virus-free amplicon packaging methods were developed by providing a packaging-deficient helper virus genome via a set of five overlapping cosmids (Fraefel et al., J. Virol. 70:7190-7197, 1996; see also U.S. Pat. No. 5,998,208) or by using a bacterial artificial chromosome (BAC) that encodes for the entire HSV genome minus its cognate cleavage/packaging Stavropoulos and Strathdee, J. Virol. 72:7137-7143, 1998; Saeki et al., Hum Gene Ther. 9:2787-2794, 1998). Such cosmids can be used as the packaging vector in the methods described herein. Thus, the packaging vector can be a cosmid-based vector or a set of vectors including cosmid-based vectors that are prepared so that none of the viral particles used will contain a functional herpesvirus cleavage-packaging site containing sequence. This sequence, which is not encoded by the packaging vector(s), is referred to as the “a” sequence. The “a” sequence can be deleted from the packaging vector(s) by any of a variety of techniques practiced by those of ordinary skill in the art. For example, one can simply delete the entire sequence (by, for example, the techniques described in U.S. Pat. No. 5,998,208). Alternatively, one can delete a sufficient portion of the sequence to render it incapable of packaging. Another alternative is to insert nucleotides into the site that render it non-functional.

The core of the herpes virus particle is formed from a variety of structural genes that create the capsid matrix. It is necessary to have those genes for matrix formation present in a susceptible cell used to prepare particles. Preferably, the necessary envelope proteins are also expressed. In addition, there are a number of other proteins present on the surface of a herpes virus particle. Some of these proteins help mediate viral entry into certain cells, and as this is known to those of ordinary skill in the art, one would know to alter the sequences expressed by the viral particle in order to alter the cell type the viral particle infects or improve the efficiency with which the particle infects a natural cellular target. Thus, the inclusion or exclusion of the functional genes encoding proteins that mediate viral entry into cells will depend upon the particular use of the particle.

In addition to a packaging vector, the herpes virus amplicon systems described herein include an amplicon plasmid. The amplicon plasmid contains a herpes virus cleavage/packaging site containing sequence, an origin of DNA replication (ori) that is recognized by the herpes virus DNA replication proteins and enzymes, and a transgene of interest (e.g., a nucleic acid sequence that encodes a therapeutically effective protein). This vector permits packaging of desired nucleotide inserts in the absence of helper viruses. In some embodiments, the amplicon plasmid contains at least one heterologous DNA sequence that is operatively linked to a promoter sequence (we discuss promoter and other regulatory sequences further below). More specifically, the amplicon plasmid can contain one or more of the following elements: (1) an HSV-derived origin of DNA replication (ori) and packaging sequence (“a” sequence); (2) a transcription unit driven typically by the HSV-1 immediate early (IE) 4/5 promoter followed by an SV-40 polyadenylation site; and (3) a bacterial origin of replication and an antibiotic resistance gene for propagation in E. coli (Frenkel, supra; Spaete and Frenkel, Cell 30:295-304, 1982).

Amplicon plasmids are dependent upon helper virus function to provide the replication machinery and structural proteins necessary for packaging amplicon plasmid DNA into viral particles. Helper packaging function is usually provided by a replication-defective virus that lacks an essential viral regulatory gene. The final product of helper virus-based packaging contains a mixture of varying proportions of helper and amplicon virions. Recently, helper virus-free amplicon packaging methods were developed by providing a packaging-deficient helper virus genome via a set of five overlapping cosmids (Fraefel et al., J. Virol. 70:7190-7197, 1996) or by using a bacterial artificial chromosome (BAC) that encodes for the entire HSV genome minus its cognate cleavage/packaging signals (Stavropoulos and Strathdee, J. Virol. 72:7137-7143, 1998; Saeki et al., Hum. Gene Ther. 9:2787-2794, 1998).

Methods for generating helper virus-free Herpesvirus amplicons: Generally, the methods of the invention are carried out by transfecting a host cell with several vectors and then isolating HSV amplicon particles produced by the host cell (while the language used herein may commonly refer to a cell, it will be understood by those of ordinary skill in the art that the methods can be practiced using populations (whether substantially pure or not) of cells or cell types, examples of which are provided elsewhere in our description). The method for producing an hf-HSV amplicon particle can be carried out, for example, by co-transfecting a host cell with: (i) an amplicon vector comprising an HSV origin of replication, an HSV cleavage/packaging signal, and a heterologous transgene expressible in a cell; (ii) one or more vectors that, individually or collectively, encode all essential HSV genes but exclude all cleavage/packaging signals; and (iii) a vhs expression vector encoding a virion host shutoff protein. One can then isolate or purify (although absolute purity is not required) the HSV amplicon particles produced by the host cell. When the HSV amplicon particles are harvested from the host cell medium, the amplicon particles are substantially pure (i.e., free of any other virion particles) and present at a concentration of greater than about 1×10⁶ particles per milliliter. To further enhance the use of the amplicon particles, the resulting stock can also be concentrated, which affords a stock of isolated HSV amplicon particles at a concentration of at least about 1×10⁷ particles per milliliter.

The amplicon vector can either be in the form of a set of vectors or a single bacterial-artificial chromosome (“BAC”), which is formed, for example, by combining the set of vectors to create a single, doublestranded vector. As noted above, methods for preparing and using a five cosmid set are disclosed in, for example, Fraefel et al. (J. Virol., 70:7190-7197, 1996), and methods for ligating the cosmids together to form a single BAC are disclosed in Stavropoulos and Strathdee (J. Virol. 72:7137-43, 1998). The BAC described in Stavropoulos and Strathdee includes apac cassette inserted at a BamHI site located within the UL41 coding sequence, thereby disrupting expression of the HSV-1 virion host shutoff protein.

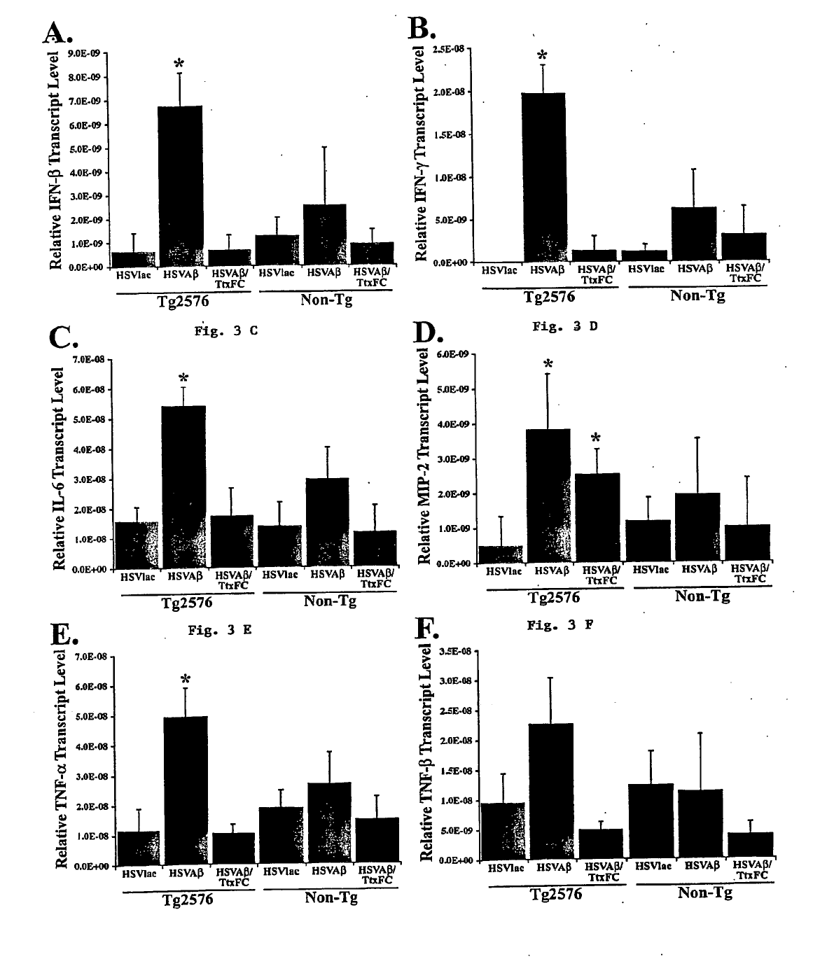

By “essential HSV genes”, it is intended that the one or more vectors include all genes that encode polypeptides that are necessary for replication of the amplicon vector and structural assembly of the amplicon particles. Thus, in the absence of such genes, the amplicon vector is not properly replicated and packaged within a capsid to form an amplicon particle capable of adsorption. Such “essential HSV genes” have previously been reported in review articles by Roizman (Proc. Natl. Acad. Sci. USA 11:307-113, 1996; Acta Viroloeica 43:75-80, 1999). Another source for identifying such essential genes is available at the Internet site operated by the Los Alamos National Laboratory, Bioscience Division, which reports the entire HSV-1 genome and includes a table identifying the essential HSV-1 genes. The genes currently identified as essential are listed in FIG. 3.

In other embodiments, a helper-free herpesvirus amplicon particle (e.g., an hf-HSV) can be generated by: (1) providing a cell that has been stably transfected with a nucleic acid sequence that encodes an accessory protein (alternatively, a transiently transfected cell can be provided); and (2) transfecting the cell with (a) one or more packaging vectors that, individually or collectively, encode one or more (and up to all) HSV structural proteins but do not encode a functional herpesvirus cleavage/packaging site and (b) an amplicon plasmid comprising a sequence that encodes a functional herpesvirus cleavage/packaging site and a herpesvirus origin of DNA replication (ori). The amplicon plasmid described in (b) can also include a sequence that encodes a therapeutic agent. In another embodiment, the method comprises transfecting a cell with (a) one or more packaging vectors that, individually or collectively, encode one or more HSV structural proteins (e.g., all HSV structural proteins) but do not encode a functional herpesvirus cleavage/packaging site, (b) an amplicon plasmid comprising a sequence that encodes a functional herpesvirus cleavage/packaging site, a herpesvirns origin of DNA replication, and a sequence that encodes an immunomodulatory protein (e.g., an immunostimulatory protein), a tumor-specific antigen, an antigen of an infectious agent, or a therapeutic agent (e.g., a growth factor), and (c) a nucleic acid sequence that encodes an accessory protein.

The HSV cleavage/packaging signal can be any cleavage/packaging that packages the vector into a particle that is capable of adsorbing to a cell (the cell being the target for transformation). A suitable packaging signal is the HSV-I “a” segment located at approximately nucleotides 127-1132 of the a sequence of the HSV-I virus or its equivalent (Davison et al., J. Gen. Virol. 55:315-331, 1981).

The HSV origin of replication can be any origin of replication that allows for replication of the amplicon vector in the host cell that is to be used for replication and packaging of the vector into HSV amplicon particles. A suitable origin of replication is the HSV-I “c” region, which contains the HSV- I ori segment located at approximately nucleotides 47-1066 of the HSV-I virus or its equivalent (McGeogh et al., Nucl. Acids Res. 14:1727-1745, 1986). Origin of replication signals from other related viruses (e.g., HSV-2 and other herpes viruses, including those listed above) can also be used.

The amplicon plasmids can be prepared (in accordance with the requirements set out herein) by methods known in the art of molecular biology. Empty amplicon vectors can be modified by introducing, at an appropriate restriction site within the vector, a complete transgene (including coding and regulatory sequences). Alternatively, one can assemble only a coding sequence and ligate that sequence into an empty amplicon vector or one that already contains appropriate regulatory sequences (promoter, enhancer, polyadenylationi signal, transcription terminator, etc.) positioned on either side of the coding sequence. Alternatively, when using the pHSVlac vector, the LacZ sequence can be excised using appropriate restriction enzymes and replaced with a coding sequence for the transgene. Conditions appropriate for restriction enzyme digests and DNA ligase reactions are well known in the art (see, e.g., Sambrook et al., Molecular Cloning: A Laboratory Manual, Cold Spring Laboratory, Cold Spring Harbor, New York (1989); Ausubel et al. (Eds.), Current Protocols in—Molecular Biology, John Wiley & Sons, New York, N.Y., 1999 and preceding editions; and U.S. Pat. No. 4,237,224).

The amplicon systems featured in these methods and others described herein can all be modified so that the transgene carried by the amplicon plasmid is inserted into the genome of the host cell. Accordingly, the methods described herein can each include an additional step of introducing, into the host cell, a vector (which can be, but is not necessarily, a plasmid) that encodes an enzyme that mediates insertion of the transgene into the genome (this vector may be referred to herein as “an integration vector”). The integration vector can be applied to a host cell in vivo or in culture at the same time that one or more of the components of the amplicon system (e.g. the packaging vector or amplicon plasmid) are administered to the host cell. The enzyme encoded by the integration vector can be a transposase, such as that encoded by sleeping beauty or a biologically active fragment or mutant thereof (i.e., a fragment or mutant of the sleeping beauty sequence that facilitates integration of the transgene into the genome at a rate or to an extent that is comparable to that achieved when wild type sleeping beauty is used). As this system represents a fundamental advance over those in which the amplicon plasmid is maintained outside the genome (and is therefore “diluted out” as cells divide), it has broad application. Methods in which an integration vector is used in the context of an amplicon system, particularly including the hf-HSV systems described herein, can be carried out to treat patients with a wide variety of diseases or disorders associated with damage to nerves or neural cells (here, as in the methods described above, a “patient” is not limited to a human patient but can be any other type of mammal). For example, the patient can have damage to the spinal cord, Alzheimer's disease, or learning or memory deficiencies. Any of the specific types diseases or disorders involving nerve or neural cell damage (e.g., spinal cord injury, Alzheimer's disease, learning or memory deficiencies) set out herein can be treated.

In addition, one can further modify the amplicon system to improve the safety of treatments in which an integration vector is administered. Frequent transposition events may lead to mutagenesis of the host genome and, possibly, even to proto-oncogene activation (although there is no evidence that this will occur or is likely to occur; it is speculated that the amplicon might enhance the frequency of such events, as 10-15 copies of the transgenon are present within a single virion). To regulate the transposase component of the system more tightly, one could, for example, incorporate the Sleeping Beauty protein into the virion in the form of a fusion with an HSV tegument protein. Alternatively, one could effect exogenous application of transposase protein with the transgenon-containing amplicon vector. Both approaches would prevent continued synthesis of Sleeping Beauty and thus, obviate additional catalysis of transposition. In yet another strategy, one could incorporate protein instability sequences into the open reading frame to limit transposase half-life. The transposon in the integration vector should be compatible with sequences flanking the transgene in the amplicon plasmid. For example, where the transposon is of the Sleeping Beauty system, the amplicon vector can include a transgene (for integration) flanked by the Sleeping Beauty terminal repeats. Integrating forms of the HSV amplicon vector platform have been described previously. One form consists of an HSV amplicon backbone and adeno-associated virus (AAV) sequences required for integration.

The amplicon vector used in any of the methods described herein can also include a sequence that encodes a selectable marker and/or a sequence that encodes an antibiotic resistance gene. Selectable marker genes are known in the art and include, without limitation, galactokinase, beta-galactosidase, chloramphenicol acetyltransferase, beta lactamase, green fluorescent protein (GFP), alcaline phosphate, etc. Antibiotic resistance genes are also known in the art and include, without limitation, ampicillin, streptomycin, spectromycin, etc. A number of suitable empty amplicon vectors have previously been described in the art including, without limitation, pHSVIac (ATCC Accession 40544; U.S. Pat. No. 5,501,979; Stavropoulos and Strathdee, J. Virol., 72:7137-43, 1998), and pHENK (U.S. Pat. No. 6,040,172). The pHSVIac vector includes the HSV-1 a segment, the HSV-1c region, an ampicillin resistance marker, and an E. coli lacZ marker. The pHENK vector includes the HSV-1 a segment, an HSV-1 ori segment, an ampicillin resistance marker, and an E. coli LacZ marker under control of the promoter region isolated from the rat preproenkephalin gene (i.e., a promoter operable in brain cells). The sequences encoding a selectable marker, the sequences encoding the antibiotic resistance gene (which may also serve as a selectable marker), and the sequences encoding the transgene, may be under the control of regulatory sequences such as promoter elements that direct the initiation of transcription by RNA polymerase, enhancer elements, and suitable transcription terminators or polyadenylation signals. Preferably, the promoter elements are operable in the cells of the patient that are targeted for transformation. A number of promoters have been identified that are capable of regulating expression within a broad range of cell types. These include, without limitation, HSV immediate-early 4/5 (IE4/5) promoter, cytomegalovirus (“CMV”) promoter, SV40 promoter, and P-actin promoter. Likewise, a number of other promoters have been identified that can regulate expression within a narrow range of cell types. These include, without limitation, the neural-specific enolase (NSE) promoter, the tyrosine hydroxylase (TH) promoter, the GFAP promoter, the preproenkephalin (PPE) promoter, the myosin heavy chain (MHQ promoter), the insulin promoter, the cholineacetyltransferase (ChAT) promoter, the dopamine β-hydroxylase (DBH) promoter, the calmodulin dependent kinase (CamK) promoter, the c-fos promoter, the cjun promoter, the vascular endothelial growth factor (VEGF) promoter, the erythropoietin (EPO) promoter, and the EGR-I promoter. The transcription termination signal should, likewise, be operable in the cells of the patient that are targeted for transformation. Suitable transcription termination signals include, without limitation, polyA signals of HSV genes such as the vhs polyadenylation signal, SV40 poly-A signal, and CW IE1 polyA signal.

Applying the information above in effective gene therapies for neural damage has been hampered by the lack of a safe and reliable vector that can be used to transduce nerve cells. Nerve cells are effectively post-mitotic. Although both retroviral and adenoviral vectors have been employed in different clinical trials for gene therapy, both systems exhibit limitations (Uckert and Walther, Pharmacol. Ther. 63:323-347, 1994; Vile et al., Mol. Biotechnol. 5:139-158, 1996; Collins, Ernst Schering Research Foundation Workshop, 2000; Hift et al., Adv. Pharmacol. 40:137-206, 1997; Kochanek, Hum. Gene Ther. 10:2451-2459, 1999). For example, the low levels of integrin receptors for adenovirus on CLL cells mandates the use of very high adenovirus titers, preactivation of the CLL cell with IL-4 and/or anti-CD40/CD40L (Cantwell et al., Blood 88:4676-4683, 1996; Huang et al., Gene Ther. 4:1093-1099, 1997), or adenovirus modification with polycations to achieve clinically meaningful levels of transgene expression (Howard et al., Leukemia 13:1608-1616, 1999).

HSV amplicon particles can be used to transduce nerve cells (e.g., mouse, rat, human, or other mammalian). Vectors can be constructed to encode β-galactosidase (by inclusion of the lacZ gene), B7.1 (also known as CD80), or CD40L (also known as CD154), and they can be packaged using either a standard helper virus (HSVlac, HSVB7.1, and HSVCD40L) or by a helper virus-free method (hf-HSVlac, hf-HSVB7.1, and hf-HSVCD40L). Cells transduced with these vectors have been studied for their expression of heterologous genes. High rates of expression in these studies have indicated that this means of gene therapy is an efficacious and reliable means of delivering heterologous genes. These studies support the conclusion that HSV amplicons are efficient vectors for gene therapy, particularly of neurons, and that helper virus-free amplicon preparations are well suited for use in therapeutic compositions.

Therapeutic Agents: As noted, the hf-HSV amplicon particles described herein (and the cells that contain them) can express a heterologous protein (i.e., a full-length protein or a portion thereof (e.g., a functional domain or antigenic peptide) that is not naturally encoded by a herpesvirus). The heterologous protein can be any protein that conveys a therapeutic benefit on the cells in which it, by way of infection with an hf-HSV amplicon particle, is expressed or a patient who is treated with those cells.

When used for gene therapy, the transgene encodes a therapeutic transgene product, which can be either a protein or an RNA molecule.

Therapeutic RNA molecules include, without limitation, antisense RNA, inhibitory RNA (siRNA), and an RNA ribozyme. The RNA ribozyme can be either cis or trans acting, either modifying the RNA transcript of the transgene to afford a functional RNA molecule or modifying another nucleic acid molecule. Exemplary RNA molecules include, without limitation, antisense RNA, ribozymes, or siRNA to nucleic acids for huntingtin, alpha synuclein, scatter factor, amyloid precursor protein, p53, VEGF, etc..

Therapeutic proteins include, without limitation, Aβ, Aβ/TtxFC, TtxFC (and other molecular adjuvants capable of assisting in expansion and maturation of humoral immune responses; see (Lu et al., Infect Immun 62(7):2754-60, 1994) (as noted above, any of the compositions of the present invention, or methods in which they are used, can include biologically active (e.g., therapeutically useful) antigenic fragments or variants (e.g., substitution, deletion, or addition mutants) of Aβ, Aβ/TtxFC, TtxFC (and other molecular adjuvants capable of assisting in expansion and maturation of humoral immune responses), or other therapeutic proteins.

Formulation and Administration of hf-HSV amplicon particles: The hf-HSV amplicon particles described herein can be administered to patients directly or indirectly; alone or in combination with other therapeutic agents; and by any route of administration. For example, the hf-HSV amplicon particles can be administered to a patient indirectly by administering cells transduced with the vector to the patient. Alternatively, or in addition, an hf-HSV amplicon particle could be administered directly. For example, an hf-HSV amplicon particle that expresses an HSVβ/TtxFC protein can be introduced into spinal cord tissue by, for example, introducing the vector into the tissue or into the vicinity of the tissue. Amplicon particles are described in the art; specific teachings regarding the manufacture and use of HSV amplicons can be found in U.S. Ser. Nos. 09/997,848 and 10/296,551. These patent applications, and any patent applications related to them by a claim of priority, are hereby incorporated by reference in the present patent application in their entirety.

Administration of HSV protein amplicons encoding HSVAβ/TtxFC provide therapeutic benefits in the form of prevention or lessening of symptoms of Alzheimer's disease, while not causing inflammation. The helper virus-free HSV vectors disclosed herein can be administered in the same manner.

The herpesvirus amplicon particles described herein, and cells that contain them, can be administered, directly or indirectly, with other species of HSV-transduced cells (e.g., HSVAβ and HSVAβ/TtxFC transduced cells) or in combination with other therapies. Such administrations may be concurrent or they may be done sequentially. Thus, in one embodiment, HSV amplicon particles, the vectors with which they are made (i.e., packaging vectors, amplicon plasmids, and vectors that express an accessory protein) can be injected into a living organism or patient (e.g., a human patient) to treat, for example, spinal cord damage or Alzheimer's disease. In further embodiments, one or more of these entities can be administered after administration of a therapeutically effective amount of another substance.

The concentrated stock of HSV amplicon particles is effectively a composition of the HSV amplicon particles in a suitable carrier. HSV amplicon particles can also be administered in injectable dosages by dissolving, suspending, or emulsifying them in physiologically acceptable diluents with a pharmaceutical carrier (at, for example, about 1×10⁷ amplicon particles per ml). Such carriers include sterile liquids, such as water and oils, with or without the addition of a surfactant and other pharmaceutically and physiologically acceptable carriers, including adjuvants, excipients or stabilizers. The oils that can be used include those obtained from animals or vegetables, petroleum based oils and synthetic oils. For example, the oil can be a peanut, soybean, or mineral oil. In general, water, saline, aqueous dextrose and related sugar solutions, glycols (e.g., propylene glycol or polyethylene glycol) are preferred liquid carriers, particular when the amplicon particles are formulated for administration by injection.

For use as aerosols, the HSV amplicon particles, in solution or suspension, can be packaged in a pressurized aerosol container together with suitable propellants, for example, hydrocarbon propellants like propane, butane, or isobutene with conventional adjuvants. The particles can also be administered in a non-pressurized form such as in a nebulizer or atomizer.

Other Methods of Administration: In addition to gene therapy (e.g., using hf-HSV amplicons), the invention also includes administration of Aβ, Aβ/TtxFC, TtxFC (and other molecular adjuvants capable of assisting in expansion and maturation of humoral immune responses), or other therapeutic proteins by other methods. These methods include direct injection of amplicon particles, nucleic acids or the polypeptides they encode into a target tissue or a fluid that contacts the target tissue (e.g., where the target tissue is within the brain, the amplicon particle can be injected into cerebrospinal fluid), introduction of cells transduced by a nucleic acid or polypeptide of interest into target tissue (or, similarly, a fluid that contacts the target tissue), bombardment at high velocity of target tissue with amplicon particles, nucleic acids or polypeptides of interest, enhancing endogenous expression of one or more of the polypeptides of interest, as well as various other methods known to those of skill in the art. These methods are united by the result: delivery of therapeutically effective amounts of HSVAβ/TtxFC to a targeted tissue (e.g., brain tissue).

Methods of Treatment; Delivery To Target Tissue: The compositions of the present invention (including amplicons that express HSVAβ/TtxFC, and cells that contain them) can be used to prevent or lessen symptoms of Alzheimer's disease. A patient can be treated after they have been diagnosed with Alzheimer's disease. Alternatively, the compositions of the invention can be used to treat patients before symptoms of Alzheimer's have occurred. Thus, “treatment” can encompass prophylactic treatment.

HSV amplicon particles have been used to transduce motoneurons. The vectors can be constructed to encode β-galactosidase (by inclusion of the lacZ gene) and HSVAβ or HSVAβ/TtxFC, and they can be packaged using either a standard helper virus (e.g., HSVlac, HSVB7.1, and HSVCD40L) or by a helper virus-free method (e.g., hf-HSVlac, hf-HSVB7.1, and hf-HSVCD40L). As Examples 1-3 demonstrate, HSV amplicons are efficient vectors for gene therapy, and that helper virus-free amplicon preparations are well suited for use in therapeutic compositions.

Formulation and Administration of hf-HSV amplicon particles: The hf-HSV amplicon particles described herein can be administered to patients directly or indirectly; alone or in combination with other therapeutic agents; and by any route of administration. For example, the hf-HSV amplicon particles can be administered to a patient indirectly by administering cells transduced with the vector to the patient. Alternatively, or in addition, an hf-HSV amplicon particle could be administered directly. For example, an hf-HSV amplicon particle that expresses HSVAβ or HSVAβ/TtxFC can be introduced into target brain tissue by, for example, injecting the vector into the brain tissue or into the vicinity of the brain tissue.

While the compositions of the invention are not limited to those that exert a therapeutic benefit by any particular mechanism of action, administration of HSV amplicons encoding HSVAβ/TtxFC can alleviate or prevent the development of symptoms of Alzheimer's disease.

The herpesvirus amplicon particles described herein, and cells that contain them, can be administered, directly or indirectly, with other species of HSV-transduced cells (e.g., cells transduced with immunomodulatory agents) or in combination with other therapies. Such administrations may be concurrent or they may be done sequentially. Thus, in one embodiment, HSV amplicon particles, the vectors with which they are made (i.e., packaging vectors, amplicon plasmids, and vectors that express an accessory protein) can be injected into a living organism or patient (e.g., a human patient) to treat, for example, Alzheimer's disease. In further embodiments, one or more of these entities can be administered after administration of another therapeutically effective composition.

Testing For Successful Treatment: After treatment using the compositions or methods of the invention, it is possible to test treated patients to assess treatment success. One of skill in the neurological arts would be well aware of the appropriate tests to measure treatment success (e.g., tests of balance, fine motor skill, and cognition). For example, a patient treated for Alzheimer's disease can be assessed using standard cognitive tests of brain function (e.g., learning and memory). In addition, high-definition imaging techniques (e.g., MRI) can be used to assess directly neural response to treatment.

Kits: The invention includes kits that can be used to maintain or increase neuronal plasticity, strengthen synaptic transmission, and improve memory or learning. These kits can include all of the necessary reagents for carrying out the methods of the invention, and can include any of the compositions of the invention. In addition, kits can include detailed instructions for effective use. For example, a kit for treating Alzheimer's disease can include amplicons containing HSVAβ or HSVAβ/TtxFC, detailed instructions for administering the amplicons to the appropriate tissue, and instructions for confirming the effectiveness of amplicon therapy.

Model Organisms for Studying Alzheimer's Disease: The invention includes methods for producing model organisms (e.g., mouse, rat) useful in studying Alzheimer's disease and methods of treating it. For example, as shown in Examples 1-3, a mouse model can be produced by delivering HSVAβ or HSVAβ/TtxFC treatment to a particular strain of mouse. One of the advantages of this invention is that such an organismic model of Alzheimer's disease can be used to determine the relationship between Aβ antigen structure/context and the elicitation of protective immune responses that prevent amyloid plaque deposition and/or lead to dissolution of pre-existing amyloid pathology. Development of an immunotherapeutic approach for AD is an even more challenging endeavor given the extant inflammatory state within the afflicted brain. Employing the HSV amplicon to modulate immune responses through different routes of inoculation, co-expression of various immunomodulating factors, and design of Aβ pathogenic peptides with varying structural characteristics makes this a unique and advantageous approach to studying how to impede or reverse disease progression. This methodology, and the organismic model that makes it possible, not only affords the development of novel AD immunotherapeutics, but contributes to the mechanistic dissection of AD pathogenesis and the immune responses required to mediate protection.

### EXAMPLES

Cell Culture

Baby hamster kidney (BHK) and RR1 cell lines were maintained as previously described (Lu and Federoff, Hum. Gene. Ther. 6:421-30, 1995). The NIH-3T3 mouse fibroblast cell line was originally obtained from American Type Culture Collection and maintained in Dulbecco's modified Eagle medium plus 10% fetal bovine serum.

Amplicon Construction and Helper Virus-free Amplicon Packaging

The previously described HSVlac amplicon contains the coding sequence for E. coli β-galactosidase under the transcriptional control of the HSV immediate-early 4/5 gene promoter (Geller and Breakefield, Science 241:1667-9, 1988). The 126-bp sequence encoding Aβ1-42 was PCR-amplified using sequence-specific primers that contained Bain HI and Hind III restriction sites and cloned into the HSVPrPUC anplicon vector (Geller and Breakefield, Science 241:1667-9, 1988) to create HSVAβ. The Aβ1-42 sense primer was 5′-CCCGAAGCTTACCATGGATGCAGAATTCCGACATGACTCAGG-3′ (SEQ ID NO:1) and the Aβ1-42 sense primer was 5′-CCCGAAGCTTACCATGGATGCAGAATTCCGACATGACT-CAGG-3′ (SEQ ID NO:2). HSVAβ/TtxFC was constructed by PCR amplifying the 1356-bp tetanus toxin fragment C segment (TtxFC) using gene-specific primers that contained BamHI and Sacd restriction sites and the resultant product was cloned into the HSVAβ vaccine vector. The TtxFC sense primer was 5′-GCGGGATCCAAAAATCTGGATTGTTGGGTTGATAAT-3′ (SEQ ID NO:3) and the TtxFC antisense primer was 5′-CGACTGAGCTCTTAATCA-TTGTCCATCCTTCATCTGT-3′ (SEQ ID NO:4). The newly designed vectors were sequenced to confirm identity, and in the case of HSVAβ/TtxFC, to ensure the maintenance of translational reading frame between Aβ1-42 and TtxFC coding sequences. Amplicon stocks were prepared using a modified helper virus-free packaging method that has been described previously (Bowers et al., Gene. Ther. 8:111-120, 2001). Vector titers were determined using expression- and transduction-based methodologies (Bowers et al., Mol. Ther. 1(3):294-299, 2000).

Administration Paradigm and Serum Isolation

All animal housing and procedures were performed in compliance with guidelines established by the University Committee of Animal Resources at the University of Rochester. Four to eight week-old APP_{Swe} Tg2576 mice (Taconic, Germantown, N.Y.) and non-transgenic littermates were vaccinated via the subcutaneous route with PBS vehicle or one of the following amplicons: HSVlac, HSVAβ1-42, or HSVAβ1-42/TtxFC. The vaccination schedule consisted of three separate monthly injections. Blood was collected from the lateral tail vein one week after each injection, and then once per month for 8 subsequent months. The blood was allowed to clot, then placed at 4° C. overnight to facilitate separation of the serum from the clot. The clots were removed and the serum centrifuged at 10,000×g for 10 min. to pellet any remaining blood cells and debris. The clarified serum was transferred to a fresh tube and stored at −20° C. until analyzed by ELISA.

ELISA Analyses

Microtiter plates (Corning) were coated with 100 ng/ml amyloid b antigen (Tocris Cookson Inc., Ellisville, Mo.) in carbonate buffer, or 100 ng/ml tetanus toxin fragment C (Calbiochem, San Diego, Calif.) in PBS. Negative control wells were coated with the appropriate buffer and 0.5% BSA w/v (Sigma, St. Louis, Mo.). Plates were then incubated at 37° C. for 1 hr. Plates were subsequently washed 4 times with PBST (PBS+0.1% Tween), blocked with PBST+5% (w/v) non-fat dried milk and 0.5% (w/v) BSA (Sigma) for 15 min. at 37° C., and then incubated overnight at 4° C. The following day plates were washed 4 times with PBST followed by addition of sera, added in duplicate, at dilutions of 1:128, 1:256, and 1:512 in PBS, or positive control antibodies of rabbit anti-Aβ (1:5000; Chemicon International, Temecula, Calif.) or goat anti-tetanus toxin fragment C (1:5000; Accurate Chemical, Westbury, N.Y.) to appropriate wells. The plates were subsequently incubated for 1 hr. at 37° C., then washed 10 times with PBST and blocked for 30 min. at 37° C. Plates were washed 4 times with PBST and the appropriate secondary antibodies were added (1:2000; goat anti-rabbit horseradish peroxidase (HRP), rabbit anti-goat HRP, or rabbit anti-mouse HRP all from Jackson Laboratories, West Grove, Pa.), and plates incubated for 1 hr. at 37° C. Wells were washed 5 times with PBST and 5 ml of developer reagent (3′3′5′5′tetramethyl benzidine; Sigma) and 45 ml of phosphate citrate buffer (Sigma) was added. The plates were developed for 15 min. at 22° C., and reaction stopped with 50 ml of 2N sulfric acid. The wells were analyzed at an absorbance wavelength of 450 nm using a Bio-Rad microplate reader (Hercules, Calif.).

Antibody Isotype Analysis

Detection of antibody isotype was completed using an isotype detection kit Mouse Mono AB ID kit (Zymed Laboratories, San Francisco, Calif.) as performed by Petrushina et. al. (Neurosci Lett 338(1):5-8, 2003). Briefly 96-well microtiter plates (Corning) were coated with Aβ1-42 peptide (100 ng/μl; or 100 ng/ml; Tocris) in carbonate buffer overnight at 4° C. Endogenous peroxidase activity was quenched by treatment with 0.3% hydrogen peroxide in PBS for 30 minutes. Serum samples derived from vaccinated mice were added to wells at a dilution of 1:256, and incubated for 30 min. at 37° C. Following 4 washes with PBST 1 drop of subclass-specific, rabbit anti-mouse antibody was added to each appropriate well, and subsequently incubated for 30 min. at 37° C. according to manufacturer's instructions. Wells were washed 4 times with PBST and 50 ml of diluted HRP-conjugated, goat anti-rabbit IgG (H+L) was added to each well. After a 30-min. incubation at 37° C. and 4 washes with PBST, 5 ml of 3′3′5′5′tetramethyl benzidine (Sigma) and 45 ml of phosphate citrate buffer (Sigma) was added. The plates were developed for 15 min. at 22° C. and then quenched with 50 ml of 2N sulfuric acid. Wells were read at a wavelength of 450 nm using a Bio-Rad microplate reader.

Quantitative Real-Time RT-PCR Analysis of Pro-Inflammatory Molecule Transcripts

RNA was isolated from frozen mouse hippocampal sections with TRIzol solution (Life Technologies Inc., Carlsbad, Calif.). Isolated RNA samples were treated with DNaseI (Sigma) and extracted using a phenol:chloroform extraction and ethanol precipitation. One microgram of RNA was reverse transcribed to cDNA using AMV Reverse Transcriptase (Roche Diagnostic Corp., Basel, Switzerland) and random hexamers in a single PCR cycle of 10 min. at 25° C., 60 min. at 42° C., and 10 min. at 70° C. cDNA was stored at −20° C. until use in quantitative real-time PCR reactions. All TaqManm probes were synthesized and labeled with 5′-end FAM and 3′-end TAMRA dyes by Synthegen, LLC (Houston, Tex.). The GAPDH sense primer was 5′-ACTGGCATGGCCTTCCG-3′ (SEQ ID NO:5), the GAPDH antisense primer was 5′-CAGGCGGCACGTCAGATC-3′ (SEQ ID NO:6), and the GAPDH probe was 5′-TTCCTACCCCCAATGTGTCCGTCGT-3′ (SEQ ID NO:7). The IFN-b sense primer sequence was 5′-CCTGGAGCAGCTGAATGGAA-3′ (SEQ ID NO:8), the IFN-b antisense primer sequence was 5′-CCGTCATCTCCATAGGGATCTT-3′ (SEQ ID NO:9), and the IFN-b probe sequence was 5′-TCAACCTCACCTACAGGGCGGACTTC-3′ (SEQ ID NO:10). The IFN-g sense primer sequence was 5′-TGAACGCTACACACTGCATCTTG-3′ (SEQ ID NO:11), the IFN-g antisense primer sequence was 5′-GTTATTCAGACTTTCTAGGCTTTCAATG-3′ (SEQ ID NO: 12), and the IFN-g probe sequence was 5′-TTTGCAGCTCTTCCTCATGG-CTGTTTC-3′ (SEQ ID NO:13). The IL-6 sense primer sequence was 5′-CTGCAAGAGACTTCCATCCAGTT-3′ (SEQ ID NO:14), the IL-6 antisense primer sequence primer was 5′-AAGTAGGGAAGGCCGTGGTT-3′ (SEQ ID NO:15), and the IL-6 probe sequence was 5′-CCTTCTTGGGACTGATGCTGGT-GACA-3′ (SEQ ID NO:16). The MIP2 sense primer sequence was 5′-CAAGAACATCCAAGCTTGAGTGT-3′ (SEQ ID NO:17), the MIP2 antisense primer sequence was 5′-TTTTGACCGCCCTTGAGAGT-3′ (SEQ ID NO:18), and the MIP2 probe sequence was 5′-CCCACTGCGCCCAGACAGAAGTCAT-3′ (SEQ ID NO:19). The TNF-a sense primer sequence was 5′-TCCAGGCGGTGCCTATGT-3′ (SEQ ID NO:20), the TNF-a antisense primer sequence was 5′-CGATCACCCCGAAGTTCAGTA-3′ (SEQ ID NO:21), and the TNF-a probe sequence was 5′-CAGCCTCTTCTCATTCCTGCTTGT-GGC-3′ (SEQ ID NO:22). The TNF-b sense primer sequence was 5′-TTCCTCCCAATACCCC-TTCC-3′ (SEQ ID NO:23), the TNF-b antisense primer sequence was 5′-TGAAGTCCCGG-ATACACAGACTT-3′ (SEQ ID NO:24), and the TNF-b probe sequence was 5′-TGTGCCT-CTCCTCAGTGCGCAGA (SEQ ID NO:25). Each 25-ml PCR sample contained 2.5 ml of purified cDNA, 900 nM of each appropriate primer, 50 nM of matching probe, and 12.5 ml of 2× Applied Biosystems Master Mix. The thermocycler parameters included a 2-min. incubation at 50° C., a 10-min. denaturation step at 95° C., and 40 cycles of 95° C. for 15 sec. and 60° C. for 1 minute. Fluorescent intensity of each sample was detected automatically by the Perkin-Elmer Applied Biosystems Sequence Detector 7700 machine. Each run included a target-specific standard curve dilution series, and all results were normalized to the profiles obtained via the GAPDH primer/probe set that served as a loading control. Following the PCR run, real-time data were analyzed using Perkin-Elmer Sequence Detector Software version 1.9.1 and the standard curve values.

Imaging and Morphometric Analysis of Amyloid Deposits

Brains from Tg2576 mice and non-transgenic littermates were fixed by 4% paraformaldehyde trans-cardiac perfusions. The brains were removed, post-fixed overnight in 4% paraformaldehyde in PBS, transferred to a solution of 20% sucrose in PBS overnight, and finally transferred to a solution of 30% sucrose in PBS. Brains were coronally sectioned (30 mm) using a sliding microtome, and sections were stored in cryoprotectant until used for immunocytochemical analyses.

Aβ immunocytochemistry was performed according to previously described methods with some modifications (Morgan et al., Nature 408(6815):982-985, 2000). Briefly, brain sections were washed with PBS for 2 hours to remove the cryoprotectant, then incubated with 3% H₂O₂ in PBS for 20 minutes to quench endogenous peroxidase activity. Sections were then washed and blocked in PBS with 10% normal goat serum and 0.4% Triton X-100. The sections were subsequently incubated in PBS containing 1% normal goat serum, 0.4% Triton X-100, and the Aβ-specific antibody 6E10 (1:2000; Signet, Dedham, Mass.). The sections were washed with PBS, followed by an incubation with goat anti-mouse, HRP-conjugated secondary antibodies (Jackson Laboratories, 1:1000) in PBS containing 1% normal goat serum and 0.4% Triton X-100. The sections were developed with a nickel-enhanced DAB reagent (Vector Laboratory, Burlingame, Calif.), mounted on slides, and coverslips applied. Each slide was coded and its identity concealed from the microscope operator. Aβ-positive deposits were visualized and images captured using an Olympus AX-70 microscope equipped with a motorized stage (Olympus, Melville, N.Y.) and the MCID 6.0 Imaging software (Imaging Research, Inc.). Sections were tiled under 20× magnification such that an entire brain section could be complied as single image. Approximately 400 images were captured via the tiling function of the MCID 6.0 software. Each tiled image was then analyzed using the automated target detection mode. Target criteria were established by pixel density and target area size. The pixel density was set with an upper (brighter) and lower (darker) threshold of 0.3500 ROD density and 0.7000 ROD density, then areas were established as a spatial criteria as 50 mm² to 200 mm², 200 mm² to 500 mm², or Area>500 mm². The image was scanned and all non-plaque targets (e.g., blood vessels) which met the density and area criteria were manually removed, leaving only Aβ-containing deposits that fell into one of the three categories. This allowed the measurement of the total number of plaques and total target area scanned for each image.

Thioflavin S Histochemistry

Microtome-derived mouse brain sections (30 mm) were washed with PBS for 30 minutes to remove cryoprotectant. The sections were stained for 3 minutes with Modified Weigert's hematoxylin, and developed in running tap water for 30 seconds. Sections were washed in deionized H2O twice for 3 minutes each. Sections were subsequently soaked in 5% acid alcohol solution, washed in tap water for 30 sec., and then rinsed again in deionized H₂O twice for 3 minutes. Sections were incubated with Thioflavin S for 1 min. and washed twice for 3 minutes. each in running tap water. The stain was developed in acetic acid (50% v/v) for 15 minutes, and sections were mounted and air-dried. Sections were viewed with confocal microscopy using FITC filters.

Statistical Analyses

Data were compared by ANOVA and student T-test post hoc tests. A probability of P<0.05 was considered statistically significant.

Generation of an Aβ-Specific Immune Response

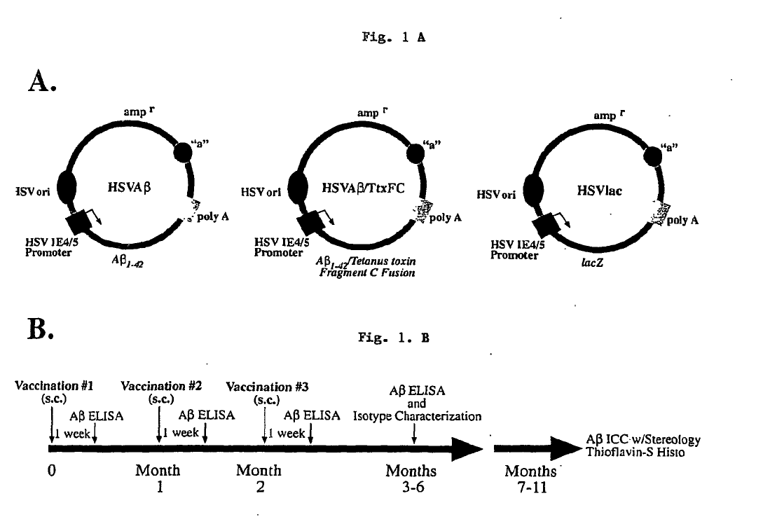

Generation of an Aβ-specific immune response in transgenic mouse AD models overexpressing human APP_{Swe} (i.e., Tg2576; (Hsiao et al., Science 274(5284):99-102, 1996)) requires a vaccination paradigm that overcomes immune tolerance. It has previously been demonstrated that amplicon vectors expressing antigens via the IE4/5 promoter are capable of transducing cells involved in antigen presentation, and, consequently, elicit antigen-specific immune responses in naive and tolerized mice (Hocknell et al., J. Virol. 76(11):5565-5580, 2002; Wang et al., Hum. Gene Ther. 13(2):261-273, 2002; Willis et al., Hum. Gene Ther. 12(15):1867-1879, 2001). To assess the feasibility of a HSV amplicon-based AD therapeutic treatment, two vectors were constructed and tested in the present study (FIG. 1A). The first amplicon expressed Aβ₁₋₄₂ alone (HSVAβ). A second amplicon vector was created that expressed Aβ₁₋₄₂ fused with the molecular adjuvant tetanus toxin fragment C (HSVAβ/TtxFC) in an effort to overcome Aβ tolerance in Tg2576 transgenic mice (Monsonego et al., Proc. Natl. Acad. Sci. USA 98(18):10273-10278, 2001), and to alter the type of immune response elicited. Fusion of TtxFC to heterologous antigens has been shown to break tolerance and assist in generation of humoral immune responses (Spellerberg et al., J. Immunol. 159(4):1885-1892, 1997). A previously described vector, designated HSVlac, expressed E. coli β-galactosidase (HSVlac) and served as a negative control vaccine (Geller and Breakefield, Science 241:1667-1669, 1988). Expression was confirmed by immunocytochemical analysis and amplicon plasmids were packaged into virions using a helper virus-free method (Bowers et al., Gene Ther. 8:111-120, 2001).

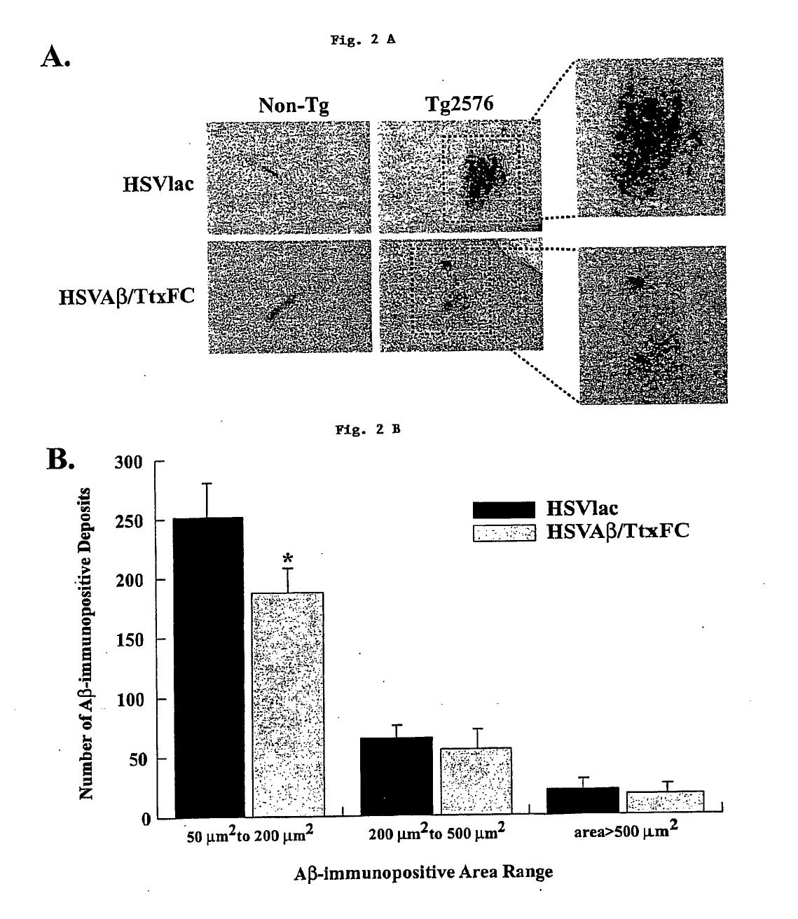

Tg2576 mice overexpress APP with the Swedish mutation (APP_{Swe}) that results in enhanced generation and extracellular deposition of the Aβ₁₋₄₂ peptide. Four to eight week-old Tg2576 mice and non-transgenic littermates received three subcutaneous (s.c.) inoculations of 1×10⁵ transduction units of one of the two vaccine vectors (HSVAβ or HSVAβ/TtxFC) or HSVlac control (see FIG. 1B for study design). Serum was collected from immunized mice one week post-vaccination and monthly thereafter. Antibodies generated to Aβ₁₋₄₂ peptide and to the fused TtxFC domain were separately assessed using ELISA (FIG. 2). Both HSVAβ- and HSVAβ/TtxFC-immunized Tg2576 mice (FIG. 2A) and non-transgenic control animals (data not shown) elicited an appreciable humoral response against Aβ₁₋₄₂, particularly detectable following the second immunization. Anti-Aβ₁₋₄₂ titers were statistically different between HSVAβ and HSVAβ/TtKFC treatment groups from 1 month post-immunization onward (P<0.001), where HSVAβ/TtxFC immunization led to a more pronounced and sustained enhancement of antibody titers. Assessment of anti-TtxFC titers indicated that the humoral responses generated by HSV amplicon-mediated immunization were antigen-specific as only HSVAβ/TtxFC-treated mice showed evidence of anti-TtxFC antibodies (FIG. 2B). These data, in aggregate, demonstrated that HSV amplicon vectors generate Aβ-specific humoral responses in the setting of Aβ tolerance and the fused TtxFC adjuvant domain markedly enhanced anti-Aβ antibody titers.

Previous Aβ peptide-based vaccination studies indicated that the elaboration of antibody isotypes arising from Th2 T-cell involvement (i.e., IgG1) were effective in preventing Aβ deposition within the brains of mice predisposed to extracellular amyloid pathology (Schenk et al., Nature 400(6740):173-177, 1999; Town et al., J. Neuroimmunol. 132(1-2):49-59, 2002). In addition, induction of Th1-related antibody isotypes (i.e., IgG2a) is indicative of the elaboration of pro-inflammatory cytokines and concomitant activation of cytotoxic T cells which could exacerbate neuronal degeneration should such a response be elicited in the CNS compartment (Furlan et al., Brain 126(Pt 2):285-291, 2003). Immune sera obtained at the 4-month post-vaccination timepoint were examined to isotype the anti-Aβ antibodies elaborated as a result of the HSVAβ and HSVAβ/TtxFC injection paradigms. Sera from HSVAβ/TtxFC-immunized Tg2576 mice possessed a significant level of anti-Aβ specific antibodies of the IgG1 isotype, indicating that the Th2 T-cell population was primarily responsible for the observed humoral response (FIG. 3). A smaller fraction of anti-Aβ antibodies elicited as a result of HSVAβ/TtxFC vaccination was of the IgA isotype. Interestingly, anti-Aβ antibodies detected in sera isolated from HSVAβ-injected Tg2576 mice were primarily of the IgM class, indicating a lack of humoral response maturation in this vaccination cohort.

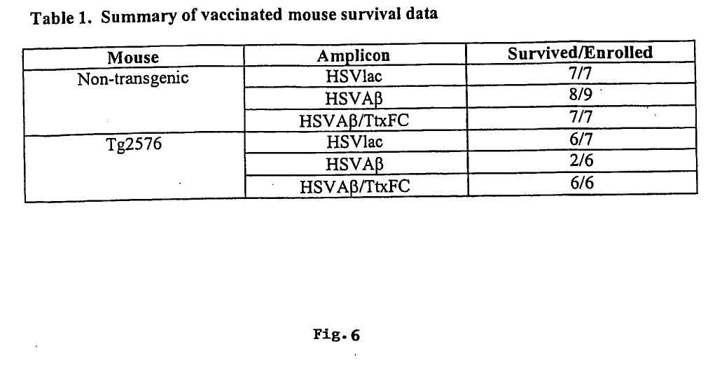

An amplicon-specific, genotype-specific mortality effect was observed in this vaccination study. Four of six Tg2576 mice receiving subcutaneous injections of the HSVAβ amplicon died approximately one week following the second vaccination (FIG. 6). Just prior to death, the four HSVAβ-injected Tg2576 mice exhibited signs of ataxia and eventually became moribund and died. One HSVAβ-vaccinated non-transgenic mouse and one HSVlac-injected Tg2576 mouse were sacrificed due to a housing cage accident. All remaining treated mice completed the study and exhibited normal behavior and weight gain. This outcome suggested that an autoimmune response had occurred in a vaccine- and genotype-specific manner due to vaccine-elicited encephalitis described previously in mice and possibly similar to that observed in clinical trial subjects (Furlan et al., Brain 126(Pt 2):285-291, 2003; Orgogozo et al., Neurology 61(1):46-54, 2003).

Comparison of Inflammation Responses

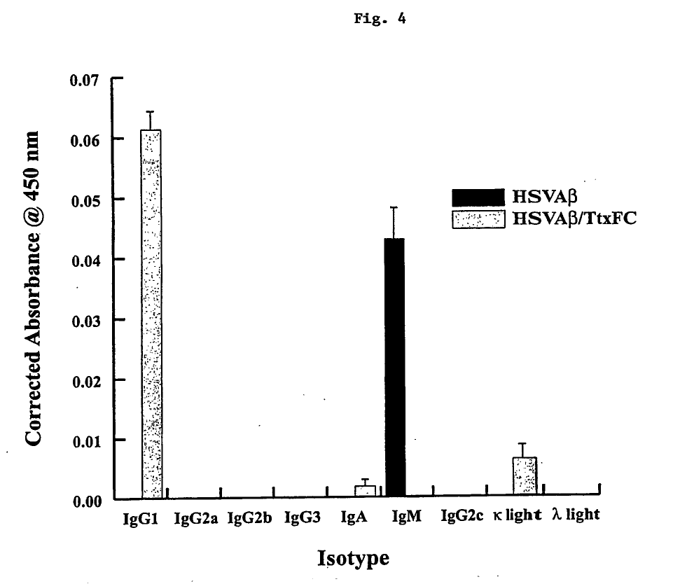

New sets of mice were given the initial two vector injections as illustrated in FIG. 1, but all mice were sacrificed within a week following the second inoculation to assess the possibility that HSVAβ selectively induces an encephalitic state in the brains of Tg2576 mice. Total RNA was prepared from microdissected hippocampus derived from each mouse and used to assess pro-inflammatory molecule transcript expression via quantitative “real-time” RT-PCR as a correlate of brain inflammation. This approach was employed previously to sensitively monitor cytokine and chemolidne transcript expression within substructures of the rodent brain (Olschowka et al., Mol. Ther. 7(2):218-227, 2003). Six pro-inflammatory molecules were selected for profiling based upon their potent activities within the brain: IFN-β, IFN-γ, IL-6, MIP-2, TNF-α, and TNF-β (FIG. 4). A majority of immunological targets that were analyzed (IFN-β, IFN-γ, IL-6, MIP-2, and TNF-α) exhibited enhanced expression specifically within hippocampi of HSVAβ-vaccinated Tg2576 mice as compared to HSVlac-injected counterparts (P<0.05). MIP-2 expression was also significantly increased in HSVAβ/TtxFC-treated Tg2576 mice (FIG. 4D). TNF-β levels trended higher but this difference did not reach statistical significance (FIG. 4F). Pro-inflammatory molecule transcript expression was at or near baseline levels in the remaining treatment/genotype groups. These results strongly suggested that HSVAβ-mediated vaccination of Tg2576 induced a vigorous inflammatory response within the brain, a condition that may have contributed to their mortality.

Amplicon Treatment and Amyloid Plaque Burden

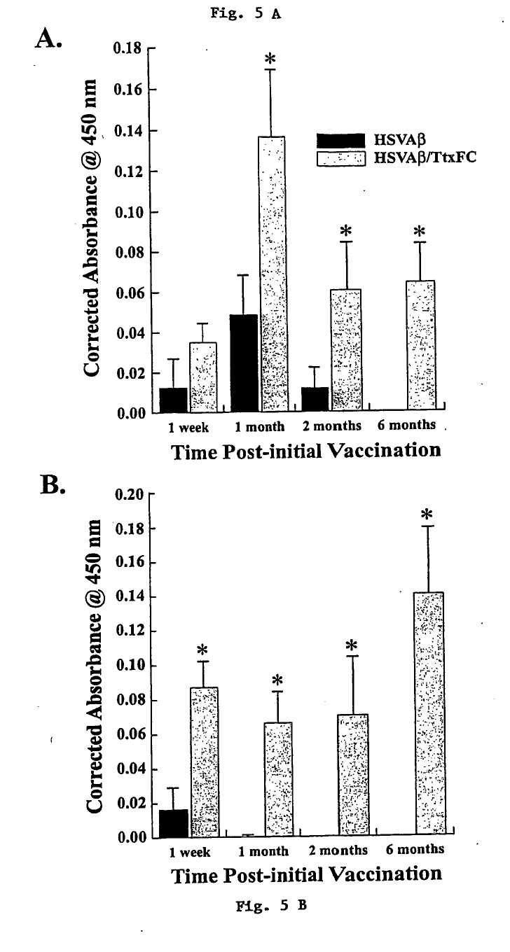

To assess the effects of amplicon treatment on amyloid plaque burden, HSVAβ/TtxFC- and HSVlac-treated mice were sacrificed at 11 months of age and brains were processed for Aβ immunohistochemistry and Thioflavin-S histochemistry. Microscopic inspection of Aβ deposits (6E10-positive) in brains obtained from the two treatment groups showed marked differences in “plaque” morphology (FIG. 5). HSVlac-immunized Tg2576 mice appeared to qualitatively harbor more Aβ deposits that were densely stained with the 6E10 antibody (FIG. 5A). Conversely, brains of HSVAβ/TtxFC-treated Tg2576 mice showed evidence of Aβ deposits that were more diffusely labeled by the 6E10 antibody. Enumeration of 6E10-positive Aβ deposits by quantitative morphometric analysis revealed differences in sizes of deposits susceptible to HSVAβ/TtxFC treatment (FIG. 5B). Deposits with areas between 50 μm² and 200 μm² were significantly reduced (P<0.05) in HSVAβ/TtxFC-treated Tg2576 mice as compared to those receiving the control treatment. The numbers of 6E10-positive deposits encompassing larger areas were not found to statistically differ between the two treatment groups. Thioflavin-S histochemistry, which stains fibrillogenic structures, also highlighted significant differences in the fibrillogenic nature of amyloid deposits between HSVlac and HSVAβ/TtxFC-treated animals. Thioflavin-S-positive deposits in brains of HSVlac-immunized Tg2576 mice appeared larger and stained more intensely than those found in HSVAβ/TtxFC-treated counterparts. These data in aggregate indicate that the HSVAβ/TtxFC treatment resulted in a highly Th2-like humoral response that imparted a significant inhibitory effect on Aβ deposition in Tg2576 mice. Moreover, treatment via this approach did not induce severe brain inflammation as was overtly evident in HSVAβ-treated Tg2576 mice.

A Human Case Study

An 83 year old woman is diagnosed by a physician as suffering from the initial stages of Alzheimer's Disease. Most strikingly, she exhibits noticeably worse memory than previously, and she has particular difficulty in remembering events that occurred in the recent past. This causes much concern among her family, and makes it more difficult for her to live the independent life to which she is used.

Under supervision of her physician, the patient is injected subcutaneously in the upper right arm with HSVAβ/TtxFC. Over the next month, the patient's memory improves noticeably, and her ability to remember events in the recent past is especially improved. Other symptoms of Alzheimer's disease are also noticeably ameliorated. Brain scans reveal a significant diminishment in the amount of amyloid plaques previously detected in her brain.

The patient's physician regularly assesses the patient, and repeats the treatment once every two to six months, depending on his assessment of his patient's progress in improving and maintaining her memory.

A number of embodiments of the invention have been described. Nevertheless, it will be understood that various modifications may be made without departing from the spirit and scope of the invention. Other embodiments may be found within the scope of the following claims.

1. A method of treating a patient with a neurodegenerative disease characterized by extracellular plaques, the method comprising administering Aβ, or an antigenic fragment or variant thereof, and a molecular adjuvant to the patient in an amount effective to improve one or more symptoms of the neurodegenerative disease.

2. The method of claim 1, wherein the molecular adjuvant is tetanus toxin Fragment C or keyhole limpet hemocyanin.

3. (canceled)

4. The method of claim 1, wherein the neurodegenerative disease is Alzheimer's disease.

5. The method of claim 1, wherein the Aβ, or antigenic fragment or variant thereof, and the molecular adjuvant are administered by injection.

6. The method of claim 1, wherein the Aβ, or antigenic fragment or variant thereof, and the molecular adjuvant are encoded by a nucleic acid.

7. The method of claim 6, wherein the nucleic acid is contained within a plasmid, expression vector, of virus or an amplicon.

8. (canceled)

9. The method of claim 7, wherein the amplicon is a herpes-simplex virus (HSV) or HSVhf (HSV helper-free) amplicon.

10. (canceled)

11. (canceled)

12. The method of claim 1, wherein Aβ and the molecular adjuvant are admixed, chemically conjugated, or fused into a recombinant polypeptide.

13. (canceled)

14. (canceled)

15. (canceled)

16. The method of claim 1, wherein the symptoms comprise impaired memory, impaired thinking, disorientation, confusion, misplacing objects, impaired abstract thinking, difficulty performing familiar tasks, changes in personality, changes in behavior, impaired judgment, impaired ability to follow directions, impaired language skills, impaired communication skills, impaired visual skills, impaired spatial skills, loss of motivation, loss of initiative, or change from normal sleep patterns.

17. The method of claim 1, wherein the method further comprises administration of a conventional adjuvant.

18. The method of claim 17, wherein the conventional adjuvant is alum.

19. A pharmaceutically acceptable composition comprising Aβ, or an antigenic fragment or variant thereof, a molecular adjuvant, and a delivery vehicle.

20. The composition of claim 19, wherein the molecular adjuvant is tetanus toxin Fragment C or keyhole limpet hemocyanin.

21. (canceled)

22. The composition of claim 19, wherein the vehicle is a virus.

23. The composition of claim 22, wherein the virus is an HSV virus.

24. The composition of claim 19, wherein the vehicle is an amplicon.

25. An isolated nucleic acid comprising a sequence encoding Aβ, or an antigenic fragment or variant thereof, and a sequence encoding a molecular adjuvant.

26. The nucleic acid of claim 25, wherein the molecular adjuvant is tetanus toxin Fragment C or keyhole limpet hemocyanin.

27. (canceled)

28. A method of treating a patient with a neurodegenerative disease characterized by extracellular plaques, the method comprising administering to the patient
(a) an amplicon plasmid comprising an HSV origin of replication, an HSV cleavage/packaging signal, and a heterologous transgene expressible in the host cell,
(b) one or more vectors that, individually or collectively, encode all essential HSV genes but exclude all cleavage/packaging signals, and
(c) a vector encoding an accessory protein, wherein the transgene encodes a therapeutic protein that improves one or more symptoms of the neurodegenerative disease.

29. The method of claim 28, wherein the neurodegenerative disease is Alzheimer's disease.

30. The method of claim 28, wherein the transgene encodes a molecular adjuvant.

31. The method of claim 28, wherein the molecular adjuvant is tetanus toxin Fragment C or keyhole limpet hemocyanin.

32. (canceled)

33. The method of claim 28, wherein the transgene encodes Aβ.

34. The method of claim 28, wherein the transgene encodes both Aβ and a molecular adjuvant.

35-48. (canceled)
